(19) United States
(12) Patent Application Publication
(10) Pub. No.: US 2022/0110862 A1
(43) Pub. Date: Apr. 14, 2022

(54) TREATMENT OF COGNITIVE DYSFUNCTION WITH PYRROLOPYRIDINE-ANILINE COMPOUNDS

(71) Applicant: NFlection Therapeutics, Inc., Wayne, PA (US)

(72) Inventors: Christopher POWALA, Wayne, PA (US); Scott PLOTKIN, Wayne, PA (US); Kavita SARIN, Wayne, PA (US); Elaine MOREFIELD, Wayne, PA (US); Jahanbanoo SHAHRYARI, Wayne, PA (US); Peter FENN, Wayne, PA (US)

(21) Appl. No.: 17/483,468

(22) Filed: Sep. 23, 2021

Related U.S. Application Data
(60) Provisional application No. 63/082,595, filed on Sep. 24, 2020.

(51) Int. Cl. A61K 9/00; A61K 31/437

(52) U.S. Cl. CPC A61K 9/0043; A61K 9/0073; A61K 31/437

(57) ABSTRACT

Provided herein are nasal spray formulations including a compound of formula (I) and methods of using these nasal spray formulations for the treatment of ADHD, or a cognitive dysfunction disease or disorder in a subject having a neurofibromatosis. In some embodiments, the neurofibromatosis is neurofibromatosis type-1, neurofibromatosis type-2, or schwannomatosis. Compounds of formula (I) are represented by

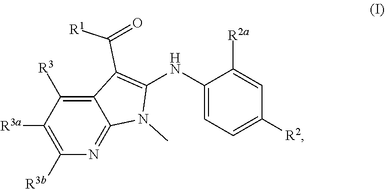

wherein R¹, R², R^{2a}, R³, R^{3a}, and R^{3b} are as defined and described herein.

### CROSS-REFERENCES TO RELATED APPLICATIONS

This application claims priority to U.S. Provisional Application No. 63/082,595 filed Sep. 24, 2020, which is incorporated in its entirety for all purposes.

### BACKGROUND OF THE INVENTION

Neurofibromatosis type 1 (NF1) occurs in approximately 1:3,500 births, and is one of the most common autosomal dominant single-gene disorders affecting neurological function in humans. Clinically, NF1 disease is characterized by the presence of benign peripheral nerve tumors, called neurofibromas, involving Schwann cells with biallelic mutations in the NF1 gene, as well as other tumor and non-tumor manifestations. See Jousma et al. Pediatr. Blood Cancer 62: 1709-1716, 2015. NF1 is associated with several dermal disorders, including dermal neurofibromas; plexiform neurofibromas; café au lait spots; and axillary and inguinal freckling. Dermal neurofibromas occur in over 95% of NF1 patients, and can appear anywhere on the body, causing itching, irritation, infection, physical pain, and disfigurement. Moreover, dermal neurofibromas are associated with social isolation and anxiety.

Benign cutaneous tumors of the vascular, keratinocytic, and melanocytic compartments often occur at birth or during childhood. These lesions, referred in this application as “birthmarks”, can cause cosmetic distress, disfigurement and social anxiety. In some cases, these lesions can predispose individuals to functional impairment or future malignancies. These birthmarks can be sporadic or arise as part of an underlying neurocutaneous syndrome.

Vascular birthmarks include, for example port wine stain/capillary malformation, angiomas, lobular capillary hemangiomas, arteriovascular malformation, lymphatic malformation, vascular malformation, hemangiomas, and other angioma. Keratinocytic nevi refers to Keratinocytic epidermal nevi and nevi sebacei. Melanocytic nevi (commonly known as moles) include, for example congenital nevi, multiple lentigines (which can occur in syndromes such as LEOPARD), ephiledes (freckles), and nevus spiilus.

In addition to birthmark formation, NF1 patients can also exhibit ADHD or a cognitive dysfunction disease or disorder (e.g., ADHD, learning disabilities, and anxiety).

NF1 is caused by one or more germ line mutations in NF1, a gene that inactivates the RAS pathway. Because the NF1 gene encodes a Ras-GAP protein, NF1 loss results in high Ras-GTP. Therefore, NF1 research has focused intensively on testing inhibitors in the Ras signaling pathway, including the Ras-MAPK cascade. See Jousma et al. Pediatr. Blood Cancer 62: 1709-1716, 2015. Four distinct MAPK cascades have been identified and named according to their MAPK module. See Akinleye et al. Journal of Hematology & Oncology 6:27, 2013. MEK proteins belong to a family of enzymes that lie upstream to their specific MAPK targets in each of the four MAP kinase signaling pathways. Two of these MEK proteins, MEK1 and MEK2, are closely related and participate in this signaling pathway cascade. Inhibitors of MEK1 and MEK2 have been shown to effectively inhibit MEK signaling downstream of Ras, and thus provide a strong rationale for targeting MEK in the treatment of NF1 (Rice et al. Medicinal Chemistry Letters 3:416-421, 2012) and thus provide a rationale for targeting MEK in the treatment of birthmarks.

Although MEK inhibitors have been developed to target the birthmarks in subjects having neurofibromatosis type-1, these treatments and administration routes do not address cognitive dysfunctions that are also associated with this condition (e.g., ADHD, learning disabilities, and anxiety).

In addition to neurofibromatosis type-1 (NF1), there are two other known neuofirbrmatosis disorders: neurofibromatosis type-2 (NF2), and schwannomatosis. Schwannomatosis is the most recently identified of these three and is believe to affect about 1 in 40,000 individuals, while NF2 is believe to affect about 1 in 25,000 individuals. Like NF1, individuals having NF2 and schwannomatosis can also exhibit ADHD or a cognitive dysfunction disease or disorder (e.g., ADHD, learning disabilities, and anxiety).

As such, there remains a need in the art to develop effective therapies that can address the cognitive dysfunctions associated with subjects having neurofibromatosis type-1, neurofibromatosis type-2 or schwannomatosis. The present disclosure addresses this need and provides related advantages as well.

### BRIEF SUMMARY OF THE INVENTION

In a first aspect, the present invention provides a method of treating cognitive dysfunction in a patient having a neurofibromatosis by administering to the subject a nasal formulation of a compound represented by formula (I)

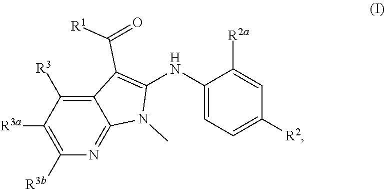

or stereoisomer, mixture of stereoisomers, and/or a pharmaceutically acceptable salt thereof, wherein R¹, R², R^{2a}, R³, R^{3a}, and R^{3b} are as defined and described herein.

In some embodiments, the neurofibromatosis is selected from the group consisting of neurofibromatosis type-1, neurofibromatosis type-2, and schwannomatosis.

In a second aspect, the present invention provides a nasal spray formulation for the treatment of cognitive disorders, including ADHD. The nasal spray formulation includes:

a compound represented by formula (I):

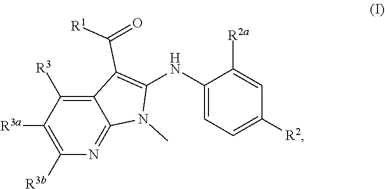

or stereoisomer, mixture of stereoisomers, and/or a pharmaceutically acceptable salt thereof, wherein R¹, R², R^{2a}, R³, R^{3a}, and R^{3b} are as defined and described herein;
and a suitable carrier, depending on formulation as a liquid nasal spray or a powdered nasal spray.

The present invention relates to intranasal compositions for treating ADHD, or a cognitive dysfunction disease or disorder, including, for example, neurodegenerative diseases or disorders and neurodevelopmental disorders such as ADHA, dementia, learning disabilities, epilepsy, etc. The compositions and methods of the present invention are formulated for intranasal delivery. In particular, nasal drug delivery of a compound of formula (I) in accordance with the present invention offers a number of advantages, including but not limited to rapid absorption, fast onset of action, avoidance of hepatic first-pass metabolism, and ease of administration.

More particularly, the compositions and methods provided herein may advantageously reduce or alleviate one or more of the core symptoms of a given neurodevelopmental disorder, for example ADHA or learning disabilities. In some aspects, the compositions and methods as provided herein may advantageously enable the compound of formula (I) to be absorbed in a sustained manner providing improved bioavailability at lower doses and/or longer duration of action. In some embodiments, the formulations and methods provided herein may provide a reduced incidence of side effects, when compared with current treatments and/or delivery methods.

Preferably, the person is in need of such treatment, and has been diagnosed with a neurofibromatosis, although the compound of formula (I) may be administered in a prophylactic sense. In some embodiments, the neurofibromatosis is selected from the group consisting of neurofibromatosis type-1, neurofibromatosis type-2, and schwannomatosis.

### BRIEF DESCRIPTION OF THE DRAWINGS

FIG. 1A and FIG. 1B show plasma and brain concentrations of Compound 1.003 in female mice following single intranasal administration of 50 μL of the nasal formulation Ex. A of Example 1. FIG. 1A: in linear scale; and FIG. 1B: in log scale.

### DETAILED DESCRIPTION OF THE INVENTION

### I. General

Although compounds of formula (I) (MEK inhibitors) have previously been described as useful in the reduction of tumor burden of persistently developing cutaneous neurofibromas (cNF) in neurofibromatosis type 1 (NF1), the present inventors have surprisingly discovered that compounds of formula (I), when administered nasally, can be useful in the treatment of ADHD, or a cognitive dysfunction disease or disorder in a subject having a neurofibromatosis. The neurofibromatosis can be neurofibromatosis type-1, neurofibromatosis type-2, or schwannomatosis.

Accordingly, provided herein are nasal spray formulations including compounds of formula (I) and methods of using these nasal spray formulations for the treatment of ADHD, or a cognitive dysfunction disease or disorder in a subject having a neurofibromatosis. The neurofibromatosis can be neurofibromatosis type-1, neurofibromatosis type-2, or schwannomatosis. The nasal spray formulations are administered, typically with a metering device to provide a specific dosage amount, effective for the treatment.

### II. Definition

The abbreviations used herein have their conventional meaning within the chemical and biological arts.

Where substituent groups are specified by their conventional chemical formulae, written from left to right, they equally encompass the substituents that would result from writing the structure from right to left, e.g., —CH₂O—is meant to include —OCH₂—.

Unless specifically indicated otherwise, compounds of formula (I) are 1-methyl-1H-pyrrolo[2,3-b]pyridine compounds, where the nitrogen (N) atom (with “*”) of the pyrrolo[2,3-b]pyridine core is substituted with methyl:

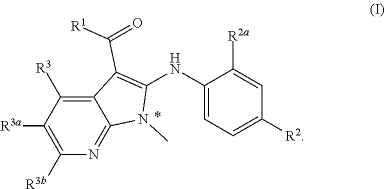

“Alkyl” refers to a straight or branched, saturated, aliphatic radical having the number of carbon atoms indicated (i.e., C₁-C₆ means one to six carbons). Alkyl can include any number of carbons, such as C₁-C₂, C₁-C₃, C₁-C₄, C₁-C₅, C₁-C₆, C₁-C₇, C₁-C₈, C₁-C₉, C₁-C₁₀, C₂-C₃, C₂-C₄, C₂-C₅, C₂-C₆, C₃-C₄, C₃-C₅, C₃-C₆, C₄-C₅, C₄-C₆ and C₅-C₆. For example, C₁-C₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc.

“Alkylene” refers to a straight or branched, saturated, aliphatic radical having the number of carbon atoms indicated (i.e., C₁-C₆ means one to six carbons), and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene.

“Alkenyl” refers to a straight chain or branched hydrocarbon having at least 2 carbon atoms and at least one double bond and having the number of carbon atom indicated (i.e., C₂-C₆ means to two to six carbons). Alkenyl can include any number of carbons, such as C₂, C₂-C₃, C₂-C₄, C₂-C₅, C₂-C₆, C₂-C₇, C₂-C₈, C₂-C₉, C₂-C₁₀, C₃, C₃-C₄, C₃-C₅, C₃-C₆, C₄, C₄-C₅, C₄-C₆, C₅, C₅-C₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl.

“Alkynyl” refers to either a straight chain or branched hydrocarbon having at least 2 carbon atoms and at least one triple bond and having the number of carbon atom indicated (i.e., C₂-C₆ means to two to six carbons). Alkynyl can include any number of carbons, such as C₂, C₂-C₃, C₂-C₄, C₂-C₅, C₂-C₆, C₂-C₇, C₂-C₈, C₂-C₉, C₂-C₁₀, C₃, C₃-C₄, C₃-C₅, C₃-C₆, C₄, C₄-C₅, C₄-C₆, C₅, C₅-C₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃-C₆, C₄-C₆, C₅-C₆, C₃-C₈, C₄-C₈, C₅-C₈, C₆-C₈, C₃-C₉, C₃-C₁₀, C₃-C₁₁, and C₃-C₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2]bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃-C₈ cycloalkyl, exemplary groups include, but are not limited to cyclopropyl, cyclobutyl, cyclopentyl, cyclohexyl, cycloheptyl and cyclooctyl.

“Cycloalkylalkyl” refers to a radical having an alkyl component and a cycloalkyl component, where the alkyl component links the cycloalkyl component to the point of attachment. The alkyl component is as defined above, except that the alkyl component is at least divalent, an alkylene, to link to the cycloalkyl component and to the point of attachment. The alkyl component can include any number of carbons, such as C₁-C₆, C₁-C₂, C₁-C₃, C₁-C₄, C₁-C₅, C₂-C₃, C₂-C₄, C₂-C₅, C₂-C₆, C₃-C₄, C₃-C₅, C₃-C₆, C₄-C₅, C₄-C₆ and C₅-C₆. The cycloalkyl component is as defined above. Exemplary cycloalkyl-alkyl groups include, but are not limited to, cyclopropylmethyl, cyclobutylmethyl, cyclopentylmethyl and cyclohexylmethyl.

“Alkoxy” refers to an alkyl group having an oxygen atom that connects the alkyl group to the point of attachment: alkyl-O—. Alkoxy groups can have any suitable number of carbon atoms, such as C₁-C₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc.

“Hydroxyalkyl” refers to an alkyl group, as defined above, where at least one of the hydrogen atoms is replaced with a hydroxy group. As for the alkyl group, a hydroxyalkyl group can have any suitable number of carbon atoms, such as C₁-C₆. As for the hydroxy group, a hydroxyalkyl group can have 1, 2, 3, or 4 hydroxy groups. “Monohydroxyalkyl” refers to a hydroxyalkyl group having one hydroxy group. “Dihydroxyalkyl” refers to a hydroxyalkyl group having two hydroxy groups. Exemplary hydroxyalkyl groups include, but are not limited to, hydroxymethyl, hydroxyethyl (where the hydroxy is in the 1- or 2-position), hydroxypropyl (where the hydroxy is in the 1-, 2- or 3-position), hydroxybutyl (where the hydroxy is in the 1-, 2-, 3- or 4-position), hydroxypentyl (where the hydroxy is in the 1-, 2-, 3-, 4- or 5-position), hydroxyhexyl (where the hydroxy is in the 1-, 2-, 3-, 4-, 5- or 6-position), 1,2-dihydroxyethyl, and the like.

“Alkoxyalkyl” refers to a radical having an alkyl component and an alkoxy component, where the alkyl component links the alkoxy component to the point of attachment. The alkyl component is as defined above, except that the alkyl component is at least divalent, an alkylene, to link to the alkoxy component and to the point of attachment. The alkyl component can include any number of carbons, such as C₁-C₂, C₁-C₃, C₁-C₄, C₁-C₅, C₁-C₆, C₂-C₃, C₂-C₄, C₂-C₅, C₂-C₆, C₃-C₄, C₃-C₅, C₃-C₆, C₄-C₅, C₄-C₆ and C₅-C₆. The alkoxy component is as defined above. Examples of the alkoxy-alkyl group include, but are not limited to, 2-ethoxy-ethyl and methoxymethyl.

“Halogen” or “halo” refers to fluoro, chloro, bromo, or iodo.

“Alcohol” refers to an alkyl group (e.g., C₂₋₆ alkyl), as defined within, having a hydroxy group attached to a carbon of the chain. For example, alcohols useful in the present invention include, but are not limited to, ethanol, propanol, isopropanol, butanol, isobutanol, tert-butanol, pentanol and hexanol, among others. Alcohols useful in the present invention are fully saturated. In some embodiments, the alcohol is C₂₋₆ alcohol.

“Alkylene glycol” refers to a compound having the formula of H—[O-alkylene]-OH, wherein the alkylene group has 2 to 6, 2 to 4, or 2 to 3 carbon atoms. In some embodiments, the alkylene glycol is a C₂₋₆ alkylene glycol. In some embodiments, the C₂₋₆ alkylene glycol is propylene glycol (1.2-propanediol).

“Di-alkylene glycol” refers to a compound having the formula of HO-(alkylene-O)₂—H, wherein the alkylene group has 2 to 6, 2 to 4, or 2 to 3 carbon atoms. In some embodiments, the di-alkylene glycol is a di-(C₂₋₆ alkylene) glycol. In some embodiments, the di-(C₂₋₆ alkylene) glycol is dipropylene glycol. Dipropylene glycol can include one or more isomers, for example 4-oxa-2,6-heptandiol, 2-(2-hydroxy-propoxy)-propan-1-ol, 2-(2-hydroxy-1-methyl-ethoxy)-propan-1-ol, and 3,3′-oxybis(propan-1-ol).

“Polyethylene glycol” refers to a polymer having the formula of HO—(CH₂CH₂O)ₙ—OH with variations in subscript “n”. Suitable polyethylene glycols may have a free hydroxyl group at each end of the polymer molecule, or may have one or more hydroxyl groups etherified with a lower alkyl, e.g., a methyl group. Also suitable are derivatives of polyethylene glycols having esterifiable carboxy groups. Polyethylene glycols useful in the present invention can be polymers of any chain length or molecular weight, and can include branching. In some embodiments, the average molecular weight of the polyethylene glycol is from about 200 to about 9000. In some embodiments, the average molecular weight of the polyethylene glycol is from about 200 to about 5000. In some embodiments, the average molecular weight of the polyethylene glycol is from about 200 to about 1500. In some embodiments, the average molecular weight of the polyethylene glycol is about 400. Suitable polyethylene glycols include, but are not limited to PEG-200, PEG-300, PEG-400, PEG-600, PEG-900, PEG-1450. The number following the “PEG” in the name refers to the average molecular weight of the polymer.

“Super refined” excipients refer to excipients that are stripped of their impurities. Super refining removes polar impurities (including primary and secondary oxidation products) from an excipient without altering its chemical composition. The removal of these impurities helps to reduce excipient-Active Pharmaceutical Ingredient (API) interaction and subsequent API degradation, thereby maintaining both the stability of the drug and the final formulation. In addition, the removal of these impurities can minimize cellular irritation, ideal for various drug administration routes. Super Refined excipients of the present invention include a super refined PEG-400 and a super refined propylene glycol.

“Super refined PEG-400” or “S.R. PEG-400” refers to a high purity grade of polyethylene glycol 400 that can enhance drug activity and formulation stability. In some embodiments, “S.R. PEG-400” has a purity of no less than about 99.5%, 99.6%, 99.7%, 99.8%, or 99.9%. In some embodiments, S.R. PEG-400 has a purity of no less than about 99.8% or 99.9%.

“Super refined propylene glycol” or “S.R. propylene glycol” refers to a highly purified propylene glycol that can enhance drug activity and composition (or formulation) stability. In some embodiments, S.R. propylene glycol has a purity of no less than about 99.5%, 99.6%, 99.7%, 99.8%, or 99.9%. In some embodiments, S.R. propylene glycol has a purity of no less than about 99.8% or 99.9%.

“Transcutol” is represented by the formula: CH₃CH₂OCH₂CH₂OCH₂CH₂OH, which has a preferred IUPAC name of 2-(2-ethoxyethoxy)ethanol. Other names for 2-(2-Ethoxyethoxy)ethanol includes diethylene glycol monoethyl ether (abbreviated as DGME or DEGEE), diethylene glycol ethyl ether (abbreviated as DEGEE), ethyldiglycol, dioxitol, 3,6-dioxa-1-octanol, Carbitol, Carbitol Cellosolve, Polysolv DE, or Dowanal DE. Transcutol includes “Transcutol P” and “Transcutol HP”.

“Transcutol P” refers to a high purity grade of 2-(2-ethoxyethoxy)ethanol. “Transcutol HP” refers to a highly purified grade of 2-(2-ethoxyethoxy)ethanol that can enhance drug activity and composition (or formulation) stability. In some embodiments, Transcutol P or HP has a purity of no less than about 99.5%, 99.6%, 99.7%, 99.8%, or 99.9%. In some embodiments, Transcutol P or HP has a purity of no less than 99.8% or 99.9%. In some embodiments, Transcutol HP has a purity of about 99.90%.

“Polysorbate” refers a type of fatty ester that results from an ethoxylated sorbitan (a polyethylene glycol derivative of sorbitol) with a fatty acid. Examples of polysorbates include Polysorbate 20 (polyoxyethylene (20) sorbitan monolaurate), Polysorbate 40 (polyoxyethylene (20) sorbitan monopalmitate), Polysorbate 60 (polyoxyethylene (20) sorbitan monostearate), and Polysorbate 80 (polyoxyethylene (20) sorbitan monooleate). Suitable polysorbates include, but are not limited to the Tween™ series (available from Uniqema), which includes Tween 20 (polyoxyethylene (20) sorbitan monolaurate), Tween 40 (polyoxyethylene (20) sorbitan monopalmitate), Tween 60 (polyoxyethylene (20) sorbitan monostearate), and Tween 80 (polyoxyethylene (20) sorbitan monooleate). Other suitable polysorbates include the ones listed in R. C. Rowe and P. J. Shesky, Handbook of pharmaceutical excipients, (2006), 5th ed., which is incorporated herein by reference in its entirety.

“Salt” refers to acid or base salts of the compounds of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

“Isomer” refers to compounds with the same chemical formula but which are structurally distinguishable. Certain compounds of the present invention possess asymmetric carbon atoms (optical centers) or double bonds; the racemates, diastereomers, geometric isomers and individual isomers are all intended to be encompassed within the scope of the present invention.

“Tautomer” refers to one of two or more structural isomers which exist in equilibrium and which are readily converted from one form to another.

“Solvate” refers to a compound provided herein or a salt thereof, that further includes a stoichiometric or non-stoichiometric amount of solvent bound by non-covalent intermolecular forces. Where the solvent is water, the solvate is a hydrate.

“Hydrate” refers to a compound that is complexed to a water molecule. The compounds of the present invention can be complexed with 12 water molecule or from 1 to 10 water molecules.

“Composition” as used herein is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present invention include, but are not limited to, binders, fillers, disintegrants, lubricants, coatings, sweeteners, flavors and colors. Pharmaceutical excipients useful in the present invention for transdermal/topical delivery include, but are not limited to, enhancers, solubilizers, antioxidants, plastisizers, thickeners, polymers, and pressure sensitive adhesives. One of skill in the art will recognize that other pharmaceutical excipients are useful in the present invention.

For any one of liquid nasal spray formulations as described herein, the content of the polyethylene glycol having an average molecular weight of from about 200 to 1500 Da (e.g., PEG-400 or a super refined PEG-400) refers to a total amount by weight including the portion from a pH adjusting solution (e.g., 0.1 M citric acid in PEG400 or a super refined PEG400) and the final Q.S. 100 (Q.S stands for quantum satis). Similarly, the content of C₁₋₃ alkyl-(OCH₂CH₂)₁₋₅—OH (e.g., 2-(2-ethoxyethoxy)ethanol or Transcutol HP) refers to a total amount by weight including the portion from a pH adjusting solution (e.g., 0.1 M citric acid in 2-(2-ethoxyethoxy)ethanol or Transcutol HP) and the final Q.S. 100. Similarly, the content of water refers to a total amount by weight including the portion from a pH adjusting solution (e.g., sodium phosphate monobasic/sodium phosphate dibasic solutions) and the final Q.S. 100.

Unless specifically indicated otherwise, a pH value of a formulation described herein refers to an apparent pH value. A nasal formulation can be an non-aqueous formulation or include water, however the formulation includes substantial amounts of other excipients (e.g., one or more absorption enhancers). Therefore, the pH value of the non-aqueous formulation or the partially aqueous solution is regarded only as an apparent pH value. According to USP chapter <791>, the apparent pH value of a non-aqueous solution or suspension or the apparent pH value of a partially aqueous solution is anticipated for variability, which may be up to approximately 1 pH unit). See USP chapter <791>, the entirety of which is incorporated herein by reference for all purposes.

“Substantially free of . . . ” refers to a formulation containing no more than 1% by weight of other excipients, such as a C₂₋₆ alcohol, a C₂₋₆ alkylene glycol, or combinations thereof, each of which is defined and described herein. Polyethylene glycol (e.g., PEG-400) and/or C₁₋₃ alkyl-(OCH₂CH₂)₁₋₅—OH (e.g., 2-(2-ethoxyethoxy)ethanol or Transcutol P or HP) contain impurities including ethylene glycol and/or diethylene glycol. When the polyethylene glycol (e.g., PEG-400) and/or C₁₋₃ alkyl-(OCH₂CH₂)₁₋₅—OH (e.g., 2-(2-ethoxyethoxy)ethanol or Transcutol P or HP) are present in a formulation, the formulation contains no more than 0.5% by weight of ethylene glycol and/or diethylene glycol as impurities. In some embodiments, when the polyethylene glycol (e.g., PEG-400) and/or C₁₋₃ alkyl-(OCH₂CH₂)₁₋₅—OH (e.g., 2-(2-ethoxyethoxy)ethanol or Transcutol P or HP) are present in a formulation, the formulation contains no more than 0.25% by weight of ethylene glycol and/or diethylene glycol as impurities.

“About” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

“Inhibition”, “inhibits” and “inhibitor” refer to a compound that prohibits or a method of prohibiting, a specific action or function.

“Administering” refers to intranasal administration to the subject.

“Treat”, “treating” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation.

“Patient” or “subject” refers to a human suffering from or prone to a disease or condition that can be treated by administration of a pharmaceutical composition as provided herein. In some embodiments, the patient is a child.

“Therapeutically effective amount” refers to an amount of a compound or of a pharmaceutical composition useful for treating or ameliorating an identified disease or condition, or for exhibiting a detectable therapeutic or inhibitory effect. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Cognitive dysfunction disease or disorder” refers to a set of conditions characterized by an impaired ability to perform high-level brain functions, which include but are not limited to, the ability to learn and remember information, organize, plan, problem-solve, focus, maintain and shift attention as necessary, understand and use language, accurately perceive the environment, and perform calculations. In some embodiments, the cognitive dysfunction is a neurodegenerative disease or disorder. In some embodiments, the cognitive dysfunction is a neurodevelopmental disorder.

“Neurodegenerative disease or disorder” refers to conditions in which the nervous system loses functions due to a degenerative change in neuronal cells. Neurodegenerative disease or disorder can be divided into two groups: conditions causing problems with movement or sensation and conditions affecting memory or related to dementia. The neurodegenerative disease may be selected from the group consisting of Alzheimer's disease, frontotemporal dementia, dementia with Lewy bodies, corticobasal degeneration, Parkinson's disease, multiple system atrophy, progressive supranuclear palsy, Huntington's disease, Alexander disease, dentato-rubro-pallido-luysian atrophy, telangiectasia, spinocerebellar ataxia, Canavan disease, Cockayne syndrome, Kennedy's disease, Krabbe disease, Machado-Joseph disease, Fronto-Temporal Dementia, Pick's disease, Sandhoff disease, Schilder's disease, Steele-Richardson-Olszewski disease, tabes dorsalis, Guillain-Barre Syndrome and peripheral neuropathies such as traumatic (nerve severing or crushing), ischemic, metabolic (diabetes, uraemia), infectious, alcoholic, iatrogenic, and genetic neuropathies Pelizaeus-Merzbacher disease, multiple sclerosis, Creutzfeldt-Jakob disease, corticobasal degeneration, amyotrophic lateral sclerosis (ALS), primary lateral sclerosis and spinal muscular atrophy, but it is not limited thereto.

“Neurodevelopmental disorder” refers to conditions characterized by abnormal neurodevelopment and/or basic biobehavioral processes, including attentional and perceptual processing, executive function, inhibitory control (e.g., sensory gating), social cognition, and communication and affiliative behaviors. In some embodiments, the neurodevelopmental disorder is a learning disability. Learning disabilities include, but are not limited to, difficulty with reading, writing, math and memory. In some embodiments, the neurodevelopmental disorder is an attention deficit disorder. Exemplified neurodevelopmental disorders include attention deficit hyperactivity disorder (ADHD), attention deficit disorder (ADD), Alper's disease, schizophrenia, obsessive-compulsive disorder (OCD), and autistic spectrum disorders. In some embodiments, the neurodevelopmental disorder is a seizure disorder such as epilepsy.

“A,” “an,” or “a(n)”, when used in reference to a group of substituents or “substituent group” herein, mean at least one. For example, where a compound is substituted with “an” alkyl or aryl, the compound is optionally substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

### III. Nasal Formulations

Provided herein are nasal spray formulations, including an active agent, a compound of formula (I) described below. As will be appreciated, a nasal spray formulation is a pharmaceutical formulation and will further include excipients, some of which can possess multiple functions. For example, a given substance may act as both a solvent and a mucosal delivery-enhancing component (e.g., mucosal delivery enhancer or absorption enhancer). Nasal spray formulations can be in a liquid form or a powdered form.

In some embodiments, the nasal spray formulation is a liquid nasal spray formulation (e.g., an aqueous solution, aqueous suspension, aqueous emulsion, non-aqueous solution, non-aqueous suspension, or non-aqueous emulsion), wherein the compound of formula (I) is completely or partially solubilized.

In some embodiments, the nasal spray formulation is a powdered nasal spray formulation wherein the compound of formula (I) is present in admixture with carrier particles.

A. Liquid Nasal Spray Formulations

In some embodiments, the nasal spray formulation is a liquid nasal spray formulation and includes the compound of formula (I) described below and one or more absorption enhancement agents; and optionally one or more agents selected from preservatives, antioxidants, pH adjustment agents, viscosity regulating agents, and stabilizing agents. In some embodiments, the nasal spray formulation further includes water.

In some embodiments, the pH of the nasal spray formulation is from about 2.0 to about 8.0. In some embodiments, the nasal spray formulation has a pH of from about 3.0 to about 7.5. In some embodiments, the nasal spray formulation has a pH of about 6.0 to about 7.0.

In some embodiments, the liquid nasal spray formulation includes the compound of formula (I), or a salt thereof, in an amount of from about 5 mg/mL to about 40 mg/mL per dose. In some embodiments, the liquid nasal spray formulation includes the compound of formula (I), or a salt thereof, in an amount of from about 0.4 mg to about 2.4 mg per dose dispensed from a device including the compound. In some embodiments, the liquid nasal spray formulation includes the compound of formula (I), or a salt thereof, in an amount of from about 0.9 mg to about 2.4 mg per dose dispensed from a device including the compound. In some embodiments, the liquid nasal spray formulation includes the compound of formula (I), or a salt thereof, in an amount of from about 0.5 mg to about 2.0 mg per dose dispensed from a device including the compound. In some embodiments, the liquid nasal spray formulation includes the compound of formula (I), or a salt thereof, in an amount of from about 0.9 mg to about 1.5 mg per dose dispensed from a device including the compound. In some embodiments, the liquid nasal spray formulation includes the compound of formula (I), or a salt thereof, in an amount of from about 0.75 mg to about 1.5 mg per dose dispensed from a device including the compound. In some embodiments, the liquid nasal spray formulation includes the compound of formula (I), or a salt thereof, in an amount of from about 0.45 mg to about 1.15 mg per dose dispensed from a device including the compound. In some embodiments, the liquid nasal spray formulation includes the compound of formula (I), or a salt thereof, in an amount of from about 1.0 mg to about 2.0 mg per dose dispensed from a device including the compound.

In some embodiments, the compound of formula (I) is present in the liquid nasal spray formulation in an amount of from about 0.005% to about 5%, from about 0.01% to about 5%, from about 0.01% to about 3%, from about 0.1% to about 3%, or from about 1% to about 3% by weight on a free salt and anhydrous basis. In some embodiments, the compound of formula (I) is present in an amount of from about 0.01% to about 3% by weight on a salt-free and anhydrous basis. In some embodiments, the compound of formula (I) is present in an amount of from about 0.1% to about 3% by weight on a salt-free and anhydrous basis. In some embodiments, the compound of formula (I) is present in an amount of from about 1% to about 3% by weight on a salt-free and anhydrous basis.

In some embodiments, Compound 1.003 is present in the liquid nasal spray formulation in an amount of from about 0.005% to about 5%, from about 0.01% to about 5%, from about 0.005% to about 3%, from about 0.01% to about 3%, from about 0.1% to about 3%, or from about 1% to about 3% by weight on a salt-free and anhydrous basis. In some embodiments, Compound 1.003 is present in an amount of from about 0.005% to about 3% by weight on a salt-free and anhydrous basis. In some embodiments, Compound 1.003 is present in an amount of from about 0.01% to about 3% by weight on a salt-free and anhydrous basis. In some embodiments, Compound 1.003 is present in an amount of from about 0.1% to about 3% by weight on a salt-free and anhydrous basis. In some embodiments, Compound 1.003 is present in an amount of from about 1% to about 3% by weight on a salt-free and anhydrous basis. In some embodiments, Compound 1.003 is present in an amount of about 0.005% by weight on a salt-free and anhydrous basis. In some embodiments, Compound 1.003 is present in an amount of about 0.01% by weight on a salt-free and anhydrous basis. In some embodiments, Compound 1.003 is present in an amount of about 0.1% by weight on a salt-free and anhydrous basis. In some embodiments, Compound 1.003 is present in an amount of about 0.25% by weight on a salt-free and anhydrous basis. In some embodiments, Compound 1.003 is present in an amount of about 0.5% by weight on a salt-free and anhydrous basis. In some embodiments, Compound 1.003 is present in an amount of about 1% by weight on a salt-free and anhydrous basis. In some embodiments, Compound 1.003 is present in an amount of about 2% by weight on a salt-free and anhydrous basis.

In some embodiments, the nasal spray formulation includes one or more absorption enhancers selected from alcohol, aprotinin, benzalkonium chloride, benzyl alcohol, capric acid, ceramides, cetylpyridinium chloride, chitosan, cyclodextrins, deoxycholic acid, decanoyl, dimethyl sulfoxide, glyceryl monooleate, glycofurol, glycofurol, glycosylated sphingosines, glycyrrhetinic acids, 2-hydroxypropyl-o-cyclodextrin, laureth-9, lauric acid, lauroyl carnitine, lysophosphatidylcholine, menthol, poloxamer 407 or F68, poly-L-arginine, polyoxyethylene-9-lauryl ether, isopropyl myristate, isopropyl palmitate, lanolin, light mineral oil, linoleic acid, menthol, myristic acid, myristyl alcohol, oleic acid, oleyl alcohol, palmitic acid, polysorbate 20, polysorbate 80, propylene glycol, polyoxyethylene alkyl ethers, polyoxylglycerides, pyrrolidone, quillaia saponin, salicylic acid, sodium salt, β-sitosterol β-D-glucoside, sodium lauryl sulfate, sucrose cocoate, taurocholic acid, taurodeoxycholic acid, taurodihydrofusidic acid, thymol, tricaprylin, triolein, and alkylsaccharides.

In some embodiments, the one or more absorption enhancers are selected from a C₂₋₆ alcohol, a polyethylene glycol, a C₂₋₆ alkylene glycol, C₁₋₃ alkyl-(OCH₂CH₂)₁₋₅—OH, or combinations thereof. In some embodiments, the one or more absorption enhancers are selected from a polyethylene glycol, a C₂₋₆ alkylene glycol, C₁₋₃ alkyl-(OCH₂CH₂)₁₋₅—OH, or combinations thereof. In some embodiments, the one or more absorption enhancers are selected from a polyethylene glycol, C₁₋₃ alkyl-(OCH₂CH₂)₁₋₅—OH, or combinations thereof.

In some embodiments, the polyethylene glycol has an average molecular weight of from about 200 to about 5000 Da. In some embodiments, the polyethylene glycol has an average molecular weight of from about 200 to about 2000 Da. In some embodiments, the polyethylene glycol has an average molecular weight of from about 200 to about 1500 Da. In some embodiments, the polyethylene glycol has an average molecular weight of from about 200 to about 900 Da. In some embodiments, the polyethylene glycol is PEG-200, PEG-300, PEG-400, PEG-600, PEG-900, PEG-1450. In some embodiments, the polyethylene glycol is PEG-400. In some embodiments, the polyethylene glycol is PEG-1450. In some embodiments, the polyethylene glycol is a mixture of PEG-400 and PEG-1450.

In some embodiments, the C₂₋₆ alcohol is ethanol. In some embodiments, the C₂₋₆ alkylene glycol is propylene glycol. In some embodiments, C₁₋₃ alkyl-(OCH₂CH₂)₁₋₅—OH is 2-(2-ethoxyethoxy)ethanol.

In some embodiments, the one or more absorption enhancers are selected from a polyethylene glycol, propylene glycol, 2-(2-ethoxyethoxy)ethanol, and combinations thereof. In some embodiments, the polyethylene glycol is PEG-400, PEG-1450, or a combination thereof. In some embodiments, the one or more absorption enhancers are selected from PEG-400, PEG-1450, propylene glycol, 2-(2-ethoxyethoxy)ethanol, and combinations thereof. In some embodiments, the one or more absorption enhancers are selected from PEG-400, 2-(2-ethoxyethoxy)ethanol, and a combination thereof.

In some embodiments, the one or more absorption enhancers include a polyethylene glycol, 2-(2-ethoxyethoxy)ethanol, or combinations thereof. In some embodiments, the one or more absorption enhancers include a polyethylene glycol and 2-(2-ethoxyethoxy)ethanol. In some embodiments, the one or more absorption enhancers are a mixture of a polyethylene glycol and 2-(2-ethoxyethoxy)ethanol. In some embodiments, the one or more absorption enhancers include a polyethylene glycol, propylene glycol, 2-(2-ethoxyethoxy)ethanol, or combinations thereof. In some embodiments, the one or more absorption enhancers include a polyethylene glycol, propylene glycol, and 2-(2-ethoxyethoxy)ethanol. In some embodiments, the one or more absorption enhancers are a mixture of a polyethylene glycol, propylene glycol, and 2-(2-ethoxyethoxy)ethanol. In some embodiments, the polyethylene glycol is PEG-400, PEG-1450, or a combination thereof. In some embodiments, the one or more absorption enhancers include PEG-400, 2-(2-ethoxyethoxy)ethanol, and a combination thereof. In some embodiments, the one or more absorption enhancers include PEG-400 and 2-(2-ethoxyethoxy)ethanol. In some embodiments, the one or more absorption enhancers are a mixture of PEG-400 and 2-(2-ethoxyethoxy)ethanol. In some embodiments, the one or more absorption enhancers include PEG-400, PEG-1450, propylene glycol, 2-(2-ethoxyethoxy)ethanol, and combinations thereof. In some embodiments, the one or more absorption enhancers include PEG-400, PEG-1450, propylene glycol, and 2-(2-ethoxyethoxy)ethanol. In some embodiments, the one or more absorption enhancers are a mixture of PEG-400, PEG-1450, propylene glycol, and 2-(2-ethoxyethoxy)ethanol.

In some embodiments, the one or more absorption enhancers are ethanol, propylene glycol, 2-(2-ethoxyethoxy)ethanol, or combinations thereof. In some embodiments, the one or more absorption enhancers include 2-(2-ethoxyethoxy)ethanol. In some embodiments, the one or more absorption enhancers are 2-(2-ethoxyethoxy)ethanol. In some embodiments, the one or more absorption enhancers include ethanol and propylene glycol. In some embodiments, the one or more absorption enhancers are a mixture of ethanol and propylene glycol. In some embodiments, the one or more absorption enhancers include ethanol, propylene glycol, and 2-(2-ethoxyethoxy)ethanol. In some embodiments, the one or more absorption enhancers are a mixture of ethanol, propylene glycol, and 2-(2-ethoxyethoxy)ethanol.

In some embodiments, the one or more absorption enhancers are present in the liquid nasal spray formulation in an amount of from about 50% to about 95% by weight. In some embodiments, the one or more absorption enhancers are present in the liquid nasal spray formulation in an amount of from about 60% to about 95% by weight. In some embodiments, the one or more absorption enhancers are present in the liquid nasal spray formulation in an amount of from about 70% to about 95% by weight. In some embodiments, the one or more absorption enhancers are present in the liquid nasal spray formulation in an amount of from about 80% to about 95% by weight. In some embodiments, the one or more absorption enhancers are present in the liquid nasal spray formulation in an amount of from about 90% to about 95% by weight.

In some embodiments, the one or more absorption enhancers are present in the liquid nasal spray formulation in an amount of from about 20% to about 60% by weight. In some embodiments, the one or more absorption enhancers are present in the liquid nasal spray formulation in an amount of from about 20% to about 70% by weight. In some embodiments, the one or more absorption enhancers are present in the liquid nasal spray formulation in an amount of from about 30% to about 70% by weight. In some embodiments, the one or more absorption enhancers are present in the liquid nasal spray formulation in an amount of from about 40% to about 70% by weight. In some embodiments, the one or more absorption enhancers are present in the liquid nasal spray formulation in an amount of from about 50% to about 70% by weight. In some embodiments, the one or more absorption enhancers are present in the liquid nasal spray formulation in an amount of from about 60% to about 70% by weight.

In some embodiments, the polyethylene glycol is present in the liquid nasal spray formulation in an amount of from about 20% to about 80% by weight. In some embodiments, the polyethylene glycol is present in the liquid nasal spray formulation in an amount of from about 30% to about 80% by weight. In some embodiments, the polyethylene glycol is present in an amount of from about 40% to about 80% by weight. In some embodiments, the polyethylene glycol is present in an amount of from about 40% to about 70%, from about 40% to about 60%, from about 50% to about 60%, or from about 60% to about 80% by weight. In some embodiments, the polyethylene glycol is present in an amount of from about 40% to about 60% by weight. In some embodiments, the polyethylene glycol is present in an amount of from about 50% to about 60% by weight. In some embodiments, the polyethylene glycol is present in an amount of from about 60% to about 80% by weight. In some embodiments, the polyethylene glycol is PEG-400. In some embodiments, the polyethylene glycol is a mixture of PEG-400 and PEG-1450.

In some embodiments, PEG-400 is present in the liquid nasal spray formulation in an amount of from about 20% to about 80% by weight. In some embodiments, PEG-400 is present in the liquid nasal spray formulation in an amount of from about 30% to about 80% by weight. In some embodiments, PEG-400 is present in an amount of from about 40% to about 80% by weight. In some embodiments, PEG-400 is present in an amount of from about 40% to about 70%, from about 40% to about 60%, from about 50% to about 60%, or from about 60% to about 80% by weight. In some embodiments, PEG-400 is present in an amount of from about 40% to about 60% by weight. In some embodiments, PEG-400 is present in an amount of from about 50% to about 60% by weight. In some embodiments, PEG-400 is present in an amount of from about 60% to about 80% by weight.

In some embodiments, C₁₋₃ alkyl-(OCH₂CH₂)₁₋₅—OH is present in the liquid nasal spray formulation in an amount of from about 20% to about 60% by weight. In some embodiments, C₁₋₃ alkyl-(OCH₂CH₂)₁₋₅—OH is present in an amount of from about 30% to about 60% by weight. In some embodiments, C₁₋₃ alkyl-(OCH₂CH₂)₁₋₅—OH is present in an amount of from about 40% to about 60% by weight. In some embodiments, C₁₋₃ alkyl-(OCH₂CH₂)₁₋₅—OH is present in an amount of from about 40% to about 50% by weight. In some embodiments, C₁₋₃ alkyl-(OCH₂CH₂)₁₋₅—OH is present in an amount of from about 20% to about 30% by weight. In some embodiments, C₁₋₃ alkyl-(OCH₂CH₂)₁₋₅—OH is 2-(2-ethoxyethoxy)ethanol.

In some embodiments, 2-(2-ethoxyethoxy)ethanol is present in the liquid nasal spray formulation in an amount of from about 20% to about 60% by weight. In some embodiments, 2-(2-ethoxyethoxy)ethanol is present in an amount of from about 30% to about 60% by weight. In some embodiments, 2-(2-ethoxyethoxy)ethanol is present in an amount of from about 40% to about 60% by weight. In some embodiments, 2-(2-ethoxyethoxy)ethanol is present in an amount of from about 50% to about 60% by weight. In some embodiments, 2-(2-ethoxyethoxy)ethanol is present in an amount of from about 20% to about 30% by weight.

In some embodiments, C₂₋₆ alkylene glycol is absent in the liquid nasal spray formulation. In some embodiments, C₂₋₆ alkylene glycol is present in the liquid nasal spray formulation in an amount of from about 5% to about 30% by weight. In some embodiments, C₂₋₆ alkylene glycol is present in an amount of from about 5% to about 20% by weight. In some embodiments, C₂₋₆ alkylene glycol is present in an amount of from about 10% to about 15% by weight. In some embodiments, C₂₋₆ alkylene glycol is propylene glycol.

In some embodiments, propylene glycol is absent in the liquid nasal spray formulation. In some embodiments, propylene glycol is present in the liquid nasal spray formulation in an amount of from about 5% to about 30% by weight. In some embodiments, propylene glycol is present in an amount of from about 5% to about 20% by weight. In some embodiments, propylene glycol is present in an amount of from about 10% to about 15% by weight.

In some embodiments, PEG-400 is a super refined PEG-400.

In some embodiments, propylene glycol is a super refined propylene glycol.

In some embodiments, 2-(2-ethoxyethoxy)ethanol is Transcutol HP. In some embodiments, 2-(2-ethoxyethoxy)ethanol is Transcutol HP having a purity of >99.90%.

In some embodiments, the liquid nasal spray formulation includes one or more absorption enhancers selected from dodecyl maltoside, benzalkonium chloride, oleic acid, or salt thereof, polysorbate 20, polysorbate 80, and sodium lauryl sulfate.

In some embodiments, the liquid nasal spray formulation includes: about 0.005% (w/v) to about 2.5% (w/v) dodecyl maltoside; about 0.001 (w/v) to about 1% (w/v) benzalkonium chloride; about 0.001 (w/v) to about 1% (w/v) oleic acid, or salt thereof; a combination of about 0.005% (w/v) to about 2.5% (w/v) dodecyl maltoside and about 0.001 (w/v) to about 1% (w/v) benzalkonium chloride; a combination of about 0.005% (w/v) to about 2.5% (w/v) dodecyl maltoside and about 0.001 (w/v) to about 1% (w/v) oleic acid, or salt thereof, or a combination of about 0.001 (w/v) to about 1% (w/v) benzalkonium chloride and about 0.001 (w/v) to about 1% (w/v) oleic acid, or salt thereof and about 0.001 to about 1% of an antioxidant (e.g. sodium metabisulfite). In some embodiments, the liquid nasal spray formulation includes: about 0.005% (w/v) to about 2.5% (w/v) dodecyl maltoside; about 0.001 (w/v) to about 1% (w/v) benzalkonium chloride; about 0.001 (w/v) to about 1% (w/v) oleic acid, or salt thereof; a combination of about 0.005% (w/v) to about 2.5% (w/v) dodecyl maltoside and about 0.001 (w/v) to about 1% (w/v) benzalkonium chloride; a combination of about 0.005% (w/v) to about 2.5% (w/v) dodecyl maltoside and about 0.001 (w/v) to about 1% (w/v) oleic acid, or salt thereof, or a combination of about 0.001 (w/v) to about 1% (w/v) benzalkonium chloride and about 0.001 (w/v) to about 1% (w/v) oleic acid, or salt thereof. In some embodiments, the liquid nasal spray formulation includes: about 0.005% (w/v) to about 0.08% (w/v) benzalkonium chloride; about 0.01% (w/v) to about 0.06% (w/v) benzalkonium chloride; or about 0.01% (w/v) to about 0.04% (w/v) benzalkonium chloride; wherein the benzalkonium chloride is the sole absorption enhancement agent in the nasal spray formulation or is in present in the formulation with one or more additional absorption enhancement agents.

In some embodiments, an antioxidant is present in the liquid nasal spray formulation. Suitable antioxidants include, but are not limited to, butylated hydroxytoluene, butylated hydroxyanisole, an ascorbyl ester, or combinations thereof. In some embodiments, the antioxidant is butylated hydroxytoluene, butylated hydroxyanisole, or a combination thereof. In some embodiments, the antioxidant is a mixture of butylated hydroxytoluene and butylated hydroxyanisole. In some embodiments, the antioxidant is an ascorbyl ester including ascorbyl palmitate. In some embodiments, the antioxidant is alpha tocopherol. In some embodiments, the antioxidant is a mixture of ascorbyl palmitate and alpha tocopherol.

In some embodiments, the antioxidant is present in the liquid nasal spray formulation in an amount of from about 0.01% to about 1% by weight. In some embodiments, the antioxidant is present in an amount of from about 0.01% to about 0.5% by weight. In some embodiments, the antioxidant is present in an amount of from about 0.01% to about 0.1% by weight. In some embodiments, the antioxidant is present in an amount of from about 0.1% to about 0.5% by weight. In some embodiments, the antioxidant is butylated hydroxytoluene. In some embodiments, the antioxidant is an ascorbyl ester including ascorbyl palmitate. In some embodiments, the antioxidant is alpha tocopherol. In some embodiments, the antioxidant is a mixture of ascorbyl palmitate and alpha tocopherol.

In some embodiments, butylated hydroxytoluene is present in the liquid nasal spray formulation in an amount of from about 0.01% to about 0.5% by weight. In some embodiments, butylated hydroxytoluene is present in an amount of from about 0.01% to about 0.1% by weight. In some embodiments, butylated hydroxytoluene is present in an amount of about 0.05% by weight. In some embodiments, the ascorbyl ester including ascorbyl palmitate is present in the liquid nasal spray formulation in an amount of from about 0.01% to about 0.1% by weight. In some embodiments, ascorbyl palmitate is present in the liquid nasal spray formulation in an amount of from about 0.01% to about 0.1% by weight. In some embodiments, ascorbyl palmitate is present in an amount of about 0.05% by weight. In some embodiments, alpha tocopherol is present in the liquid nasal spray formulation in an amount of from about 0.001% to about 0.05% by weight. In some embodiments, alpha tocopherol is present in the liquid nasal spray formulation in an amount of from about 0.001% to about 0.01% by weight. In some embodiments, alpha tocopherol is present in an amount of about 0.002% by weight.

In some embodiments, the preservative is absent in the liquid nasal spray formulation.

In some embodiments, the liquid nasal spray formulation includes a preservative. In some embodiments, the preservative, when present, is benzyl alcohol, benzalkonium chloride phenoxyethanol, or a combination thereof. In some embodiments, the preservative, when present, is benzyl alcohol. In some embodiments, the preservative, when present, is phenoxyethanol. In some embodiments, the preservative, when present, is a mixture of benzyl alcohol and phenoxyethanol. In some embodiments, the preservative, when present, is benzalkonium chloride.

In some embodiments, the preservative, when present, is in an amount of from about 0.1% to about 5% by weight. In some embodiments, the preservative, when present, is in an amount of from about 0.5% to about 2% by weight.

In some embodiments, the liquid nasal spray formulation includes pH adjustment agents. In some embodiments, the pH adjustment agent is an acid, a base, a buffer, or a combination thereof. In some embodiments, the acid is adipic acid, ammonium chloride, citric acid, acetic acid, hydrochloric acid, lactic acid, phosphoric acid, propionic acid, sulfuric acid, or tartaric acid; the base is sodium hydroxide, sodium citrate, sodium bicarbonate, sodium carbonate; and the buffer is a phosphate buffer, acetate buffer, or citrate buffer.

In some embodiments, the liquid nasal spray formulation additionally includes a stabilizing agent. In some embodiments, the stabilizing agent is ethylenediaminetetraacetic acid (EDTA) or a salt thereof. In some embodiments, the EDTA is disodium EDTA. In some embodiments, the EDTA is present in an amount that is from about 0.001% to about 1%.

In some embodiments, the viscosity regulating agent is a component that acts as a thickener or gelling agent. Examples include, but not limited to, cellulose and cellulose derivatives thereof, such as hydroxypropyl cellulose and hydroxyethyl cellulose, polysaccharides, carbomers, acrylic polymers, such as Carbopol, polyvinyl alcohol and other vinylic polymers, povidone, Co-Polyvidone (Kollidon VA64) colloidal silicon dioxide, such as Aerosil® 200 or Cab-O-Sil®, such as Cab-O-Sil® M-5P, lipophilic silicon dioxide, such as Aerosil® R972, cetyl alcohols, stearic acid, glyceryl behenate, wax, beeswax, 15 petrolatum, triglycerides, lanolin and suitable mixtures thereof. In some embodiments, the viscosity regulating agent is hydroxypropyl cellulose (HPC).

In some embodiments, hydroxypropyl cellulose has an average molecular weight of about 80,000 Da, 95,000 Da, 100,000 Da, 140,000 Da, 180,000 Da, 280,000 Da, 370,000 Da, 700,000 Da, 850,000 Da, 1,000,000 Da, or 1,150,000 Da. In some embodiments, hydroxypropyl cellulose has an average molecular weight of about 140,000 Da, 180,000 Da, 280,000 Da, 370,000 Da, 700,000 Da, 850,000 Da, 1,000,000 Da, or 1,150,000 Da. In some embodiments, hydroxypropyl cellulose has an average molecular weight of about 140,000 Da, 370,000 Da, 850,000 Da, or 1,150,000 Da. In some embodiments, hydroxypropyl cellulose has an average molecular weight of from about 700,000 Da to about 1,150,000 Da.

In some embodiments, the viscosity regulating agent is a polyethylene glycol having an average molecular weight of from about 1000 to about 3000 Da. In some embodiments, the viscosity regulating agent is PEG-1000, PEG-1450, PEG-1500, PEG-2000, PEG-2500, or PEG-3000. In some embodiments, the viscosity regulating agent is PEG-1450. In some embodiments, the viscosity regulating agent is PEG-1500.

The hydroxypropyl cellulose (HPC) as described herein includes HY117, HY119, HY121, Nisso SSL, Nisso SL, Nisso L, Nisso LM, Nisso LMM, Nisso M, Nisso H, Nisso VH, Klucel ELF, Klucel EF, Klucel LF, Klucel JF, Klucel GF, Klucel MF, and Klucel HF. HY117 has an average molecular weight of about 95,000 Da; HY119 has an average molecular weight of about 370,000 Da; and HY121 has an average molecular weight of about 850,000 Da. Nisso SL has an average molecular weight of about 100,000 Da; Nisso L has an average molecular weight of about 140,000 Da; Nisso LM has an average molecular weight of about 180,000 Da; Nisso LMM has an average molecular weight of about 280,000 Da; Nisso M has an average molecular weight of about 700,000 Da; and Nisso H has an average molecular weight of about 1,000,000 Da. Suitable particle sizes of Nisso HPC (i.e., Nisso SSL, Nisso SL, Nisso L, Nisso LM, Nisso LMM, Nisso M, Nisso H, and Nisso VH) in the gel topical formulation include regular powder (about 40 mesh), fine powder (about 100 mesh), and super fine powder (about 300 mesh). See Technical date sheets of Nisso HPCs, the entirety of which is incorporated herein by reference for all purpose. Klucel EF has an average molecular weight of about 80,000 Da; Klucel LF has an average molecular weight of about 95,000 Da; Klucel JF has an average molecular weight of about 140,000 Da; Klucel GF has an average molecular weight of about 370,000 Da; Klucel MF has an average molecular weight of about 850,000 Da; and Klucel HF has an average molecular weight of about 1,150,000 Da. Suitable particle sizes of Klucel HPC in the topical formulation include regular grade and fine grade. See Technical date sheets of Klucel HPC products, the entirety of which is incorporated herein by reference for all purpose.

In some embodiments of any one of the liquid nasal spray formulations, the hydroxypropyl cellulose is Klucel JF, Klucel GF, Klucel MF, or Klucel HF. In some embodiments, the hydroxypropyl cellulose is Klucel JF, Klucel MF, or Klucel HF. In some embodiments, the hydroxypropyl cellulose is Klucel MF or Klucel HF. In some embodiments, the hydroxypropyl cellulose is Klucel JF. In some embodiments, the hydroxypropyl cellulose is Klucel GF. In some embodiments, the hydroxypropyl cellulose is Klucel MF. In some embodiments, the hydroxypropyl cellulose is Klucel HF.

In some embodiments, no viscosity regulating agent is used in the liquid nasal spray formulation.

In some embodiments, the viscosity of the liquid nasal spray formulation is no more than about 10,000 cP. In some embodiments, the viscosity of the liquid nasal spray formulation is no more than about 5,000 cP. In some embodiments, the viscosity is from about 1 cP to about 5,000 cP, from about 1 cP to about 4,000 cP, from about 1 cP to about 3,000 cP, from about 1 cP to about 2,000 cP, from about 1 cP to about 1,000 cP, or from about 1 cP to about 500 cP. In some embodiments, the viscosity is from about 1 cP to about 2,000 cP, from about 1 cP to about 1,000 cP, or from about 1 cP to about 500 cP. In some embodiments, the viscosity is from about 1 cP to about 2,000 cP. In some embodiments, the viscosity is from about 1 cP to about 1,000 cP. In some embodiments, the viscosity is from about 1 cP to about 500 cP.

In some embodiments, the liquid nasal spray formulation (A) includes:

a) the compound of formula (I);

b) PEG-400, an antioxidant, optionally a preservative, and optionally a stabilizer;

c) C₁₋₃ alkyl-(OCH₂CH₂)₁₋₅—OH; and

d) optionally a viscosity regulating agent.

In some embodiments, liquid nasal spray formulation (A) is substantially free of a C₂₋₆ alcohol, a C₂₋₆ alkylene glycol, a combination thereof, each of which is defined and described herein. In some embodiments, the liquid nasal spray formulation (A) is substantially free of ethanol, propylene glycol, diethylene glycol, or combinations thereof.

In some embodiments of liquid nasal spray formulation (A), PEG-400 is present in an amount of from about 30% to about 70%, from about 40% to about 70%, from about 40% to about 60%, from about 40% to about 50%, or from about 50% to about 60% by weight. In some embodiments, PEG-400 is present in an amount of from about 40% to about 70%, from about 40% to about 60%, or from about 50% to about 60% by weight. In some embodiments, PEG-400 is present in an amount of from about 40% to about 70% by weight. In some embodiments, PEG-400 is present in an amount of from about 40% to about 60% by weight. In some embodiments, PEG-400 is present in an amount of from about 50% to about 60% by weight. In some embodiments, PEG-400 is present in an amount of from about 50% to about 55% by weight. In some embodiments, PEG-400 is present in an amount of about 52% by weight.

In some embodiments of liquid nasal spray formulation (A), C₁₋₃ alkyl-(OCH₂CH₂)₁₋₅—OH is 2-(2-ethoxyethoxy)ethanol. In some embodiments, 2-(2-ethoxyethoxy)ethanol is present in an amount of from about 30% to about 60%, from about 40% to about 60%, or from about 40% to about 50% by weight. In some embodiments, 2-(2-ethoxyethoxy)ethanol is present in an amount of from about 40% to about 60% by weight. In some embodiments, 2-(2-ethoxyethoxy)ethanol is present in an amount of from about 40% to about 50% by weight. In some embodiments, 2-(2-ethoxyethoxy)ethanol is present in an amount of about 45% by weight.

In some embodiments of liquid nasal spray formulation (A), the antioxidant is an ascorbyl ester including ascorbyl palmitate and alpha tocopherol. In some embodiments, ascorbyl palmitate is present in an amount of from about 0.01% to about 0.1% by weight. In some embodiments, ascorbyl palmitate is present in an amount of from about 0.01% to about 0.1%, from about 0.02% to about 0.08%, or from about 0.03% to about 0.07% by weight. In some embodiments, ascorbyl palmitate is present in an amount of from about 0.03% to about 0.07% by weight. In some embodiments, ascorbyl palmitate is present in an amount of about 0.05% by weight. In some embodiments, alpha tocopherol is present in an amount of from about 0.001% to about 0.005% by weight. In some embodiments, alpha tocopherol is present in an amount of about 0.002% by weight.

In some embodiments of liquid nasal spray formulation (A), the preservative is absent.

In some embodiments of liquid nasal spray formulation (A), the stabilizing agent is ethylenediaminetetraacetic acid (EDTA) or a salt thereof. In some embodiments, the EDTA is disodium EDTA. In some embodiments, the EDTA is present in an amount that is from about 0.001% to about 1%.

In some embodiments of liquid nasal spray formulation (A), the viscosity regulating agent is absent. In some embodiments of liquid nasal spray formulation (A), the viscosity regulating agent is hydroxypropyl cellulose.

In some embodiments of liquid nasal spray formulation (A), the viscosity is from about 1 cP to about 2,000 cP, from about 1 cP to about 1,000 cP, or from about 1 cP to about 500 cP. In some embodiments, the viscosity is from about 1 cP to about 2,000 cP.

i. Mucosal Delivery Enhancer

As noted above, some excipients can provide multiple functions, or be used in a manner than allows the excipient to be characterized by more than one term. Accordingly, the liquid nasal spray formulations provided herein, will include, in some embodiments, a mucosal delivery-enhancing component. The term, “mucosal delivery-enhancing component” or mucosal delivery enhancer refers to components which enhance the release or solubility (e.g., from a formulation delivery vehicle), diffusion rate, penetration capacity and timing, uptake, residence time, stability, effective half-life, peak or sustained concentration levels, clearance and other desired mucosal delivery characteristics (e.g., as measured at the site of delivery, or at a selected target site of activity such as the bloodstream or central nervous system) of a compound(s) (e.g., biologically active compound). Enhancement of mucosal delivery can occur by any of a variety of mechanisms, including, for example, by increasing the diffusion, transport, persistence or stability of the compound, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junction physiology, reducing the viscosity of mucus overlying the mucosal epithelium, reducing mucociliary clearance rates, and other mechanisms.

Exemplary mucosal delivery enhancing components include the following: (a) an aggregation inhibitory agent; (b) a charge-modifying agent; (c) a pH control agent; (d) a degradative enzyme inhibitory agent; (e) a mucolytic or mucus clearing agent; (f) a ciliostatic agent; (g) a membrane penetration-enhancing agent selected from: (i) a surfactant; (ii) a bile salt; (ii) a phospholipid additive, mixed micelle, liposome, or carrier; (iii) an alcohol; (iv) an enamine; (v) an NO donor compound; (vi) a long-chain amphipathic molecule; (vii) a small hydrophobic penetration enhancer; (viii) sodium or a salicylic acid derivative; (ix) a glycerol ester of acetoacetic acid; (x) a cyclodextrin or beta-cyclodextrin derivative; (xi) a medium-chain fatty acid; (xii) a chelating agent; (xiii) an amino acid or salt thereof, (xiv) an N-acetylamino acid or salt thereof, (xv) an enzyme degradative to a selected membrane component; (ix) an inhibitor of fatty acid synthesis; (x) an inhibitor of cholesterol synthesis; and (xi) any combination of the membrane penetration enhancing agents recited in (i)-(x); (h) a modulatory agent of epithelial junction physiology; (i) a vasodilator agent; (j) a selective transport-enhancing agent; and (k) a stabilizing delivery vehicle, carrier, mucoadhesive, support or complex-forming species with which the compound is effectively combined, associated, contained, encapsulated or bound resulting in stabilization of the compound for enhanced nasal mucosal delivery, wherein the formulation of the compound with the intranasal delivery-enhancing agents provides for increased bioavailability of the compound in a blood plasma of a subject.

Additional mucosal delivery-enhancing agents include, for example, citric acid, sodium citrate, propylene glycol, glycerin, ascorbic acid (e.g., L-ascorbic acid), sodium metabisulfite, ethylenediaminetetraacetic acid (EDTA) disodium, benzalkonium chloride, sodium hydroxide, and mixtures thereof. For example, EDTA or its salts (e.g., sodium or potassium) are employed in amounts ranging from about 0.01% to about 2% by weight of the composition containing alkylsaccharide preservative.

B. Powdered Nasal Spray Formulations

In some embodiments, the nasal spray formulation is a powdered nasal spray formulation, including the compound of formula (I), as discussed below, and a carrier particle.

In some embodiments, the compound of formula (I) is present in the powdered nasal spray formulations in an amount of from about 0.005% to about 5%, from about 0.01% to about 5%, from about 0.01% to about 3%, from about 0.1% to about 3%, or from about 1% to about 3% by weight on a free salt and anhydrous basis. In some embodiments, the compound of formula (I) is present in an amount of from about 0.01% to about 3% by weight on a salt-free and anhydrous basis. In some embodiments, the compound of formula (I) is present in an amount of from about 0.1% to about 3% by weight on a salt-free and anhydrous basis. In some embodiments, the compound of formula (I) is present in an amount of from about 1% to about 3% by weight on a salt-free and anhydrous basis.

In some embodiments, Compound 1.003 is present in the powdered nasal spray formulations in an amount of from about 0.005% to about 5%, from about 0.01% to about 5%, from about 0.005% to about 3%, from about 0.01% to about 3%, from about 0.1% to about 3%, or from about 1% to about 3% by weight on a salt-free and anhydrous basis. In some embodiments, Compound 1.003 is present in an amount of from about 0.005% to about 3% by weight on a salt-free and anhydrous basis. In some embodiments, Compound 1.003 is present in an amount of from about 0.01% to about 3% by weight on a salt-free and anhydrous basis. In some embodiments, Compound 1.003 is present in an amount of from about 0.1% to about 3% by weight on a salt-free and anhydrous basis. In some embodiments, Compound 1.003 is present in an amount of from about 1% to about 3% by weight on a salt-free and anhydrous basis. In some embodiments, Compound 1.003 is present in an amount of about 0.005% by weight on a salt-free and anhydrous basis. In some embodiments, Compound 1.003 is present in an amount of about 0.01% by weight on a salt-free and anhydrous basis. In some embodiments, Compound 1.003 is present in an amount of about 0.1% by weight on a salt-free and anhydrous basis. In some embodiments, Compound 1.003 is present in an amount of about 0.25% by weight on a salt-free and anhydrous basis. In some embodiments, Compound 1.003 is present in an amount of about 0.5% by weight on a salt-free and anhydrous basis. In some embodiments, Compound 1.003 is present in an amount of about 1% by weight on a salt-free and anhydrous basis. In some embodiments, Compound 1.003 is present in an amount of about 2% by weight on a salt-free and anhydrous basis.

Carrier particles in the powdered nasal spray formulations described herein include any suitable excipient for powdered nasal spray formulations. Exemplary carrier particles include, but are not necessarily limited to monosaccharides such as glucose, arabinose; disaccharides such as lactose, maltose, sucrose; polysaccharides such as starch, dextrin or dextran; polyalcohols such as sorbitol, mannitol, and xylitol; and hydrates thereof. In some embodiments, monosaccharides or disaccharides are used; in another embodiment of the present invention, lactose is employed; and in still another embodiment, lactose monohydrate is used.

C. Nasal Delivery Devices

Also provided are nasal drug delivery devices including a formulation described herein. In some embodiments, the device is pre-primed. In some embodiments, the device can be primed before use. In some embodiments, the device can be actuated with one hand.

Nasal delivery is considered an attractive, safe, and easy-to-administer route for needle-free, systemic drug delivery, especially when rapid absorption and effect are desired. In addition, nasal delivery may help address issues related to poor bioavailability, slow absorption, drug degradation, and adverse events (AEs) in the gastrointestinal tract and avoids the first-pass metabolism in the liver.

Liquid nasal spray formulations can be non-aqueous or aqueous solutions, but suspensions, emulsions, liposomes, and microspheres can also be delivered. Other liquid formulations can include liposomes, microspheres, mixed aqueous-organic formulations, non-aqueous formulations, dry powder and retentive formulations (gels). In traditional spray pump systems, antimicrobial preservatives are typically required to maintain microbiological stability in liquid formulations. Metered spray pumps have dominated the nasal drug delivery market since they were introduced. The pumps typically deliver 100 μL (25-250 μL) per spray, and they offer high reproducibility of the emitted dose and plume geometry in in vitro tests.

Examples of standard metered spray pumps include those offered by Aptar Pharma, Inc., such as the multi-dose “classic technology platform” nasal spray devices, and by BD Medical-Pharmaceutical Systems, such as the Accusprayr® system. Such devices include a reservoir which holds multiple doses of the nasal spray formulation (e.g., 50, 100, 150, 200, 60, or 120 doses), a closure (e.g., screw, crimp, or snap-on), and an actuator which delivers anywhere from 45 to 1000 μL (e.g. 50, 100, 140, 150, or 200 μL) of fluid per actuation to include a single dose. The actuator may be configured to count doses, deliver gel formulations, deliver in an upside-down configuration, etc.

In traditional multi-use spray pump systems, antimicrobial preservatives are typically required to maintain microbiological stability in liquid formulations. However, preservative-free systems are also available, e.g. the Advanced Preservative Free (APF) system from Aptar, which is vented, contains a filter membrane for air flow which prevents contamination, has a metal-free fluid path for oxidizing formulations, and can be used in any orientation. Additional nasal spray devices from Aptar and others are optimized with dispenser tips that prevent clogging (useful for high-viscosity and high-volatile formulations), actuators that do not need re-priming after long periods of disuse, etc. Additional nasal spray devices are propellant driven. Yet additional nasal spray devices include dry powder inhalers.

The particle size and plume geometry can vary within certain limits and depend on the properties of the pump, the formulation, the orifice of the actuator, and the force applied. The droplet size distribution of a nasal spray is a critical parameter, since it significantly influences the in vivo deposition of the drug in the nasal cavity. The droplet size is influenced by the actuation parameters of the device and the formulation. The prevalent median droplet size should be between about 30 and about 100 μm. If the droplets are too large (>about 120 μm), deposition takes place mainly in the anterior parts of the nose, and if the droplets are too small (<about 10 μm), they can possibly be inhaled and reach the lungs and oral cavity, which should be avoided because of safety reasons. In its capacity as a surfactant, benzalkonium chloride and alkylmaltosides (e.g., a tetradecyl maltoside (TDM), a dodecyl maltoside (DDM), etc.) can affect the surface tension of droplets from a delivered nasal spray plume, producing spherical or substantially spherical particles having a narrow droplet size distribution (DSD), as well as the viscosity of a liquid formulation.

Plume geometry, droplet size and DSD of the delivered plume subsequent to spraying may be measured under specified experimental and instrumental conditions by appropriate and validated and/or calibrated analytical procedures known in the art. These include photography, laser diffraction, and impaction systems (cascade impaction, NGI). Plume geometry, droplet size and DSD can affect pharmacokinetic outcomes such as Cₘₐₓ, Tₘₐₓ, and dose proportionality.

Droplet size distribution can be controlled in terms of ranges for the D10, D50, D90, span [(D90−D10)/D50], and percentage of droplets less than 10 mm. In some embodiments, the formulation has a narrow DSD. In some embodiments, the formulation has a D(v,50) of 30-70 μm and a D(v, 90)<100 μm.

In some embodiments, the percent of droplets less than 10 μm is less than 10%. In some embodiments, the percent of droplets less than 10 μm is less than 5%. In some embodiments, the percent of droplets less than 10 μm is less than 2%. In some embodiments, the percent of droplets less than 10 μm is less than 1%.

In some embodiments, the formulation when dispensed by actuation from the device produces a uniform circular plume with an ovality ratio close to 1. Ovality ratio is calculated as the quotient of the maximum diameter (Dₘₐₓ) and the minimum diameter (Dₘᵢₙ) of a spray pattern taken orthogonal to the direction of spray flow (e.g., from the “top”). In some embodiments, the ovality ratio is less than ±2.0. In some embodiments, the ovality ratio is less than ±1.5. In some embodiments, the ovality ratio is less than ±1.3. In some embodiments, the ovality ratio is less than ±1.2. In some embodiments, the ovality ratio is less than ±1.1.

The details and mechanical principles of particle generation for different types of nasal aerosol devices has been described. See, Vidgren and Kublik, Adv. Drug Deliv. Rev. 29:157-77, 1998. Traditional spray pumps replace the emitted liquid with air, and preservatives are therefore required to prevent contamination. However, driven by the studies suggesting possible negative effects of preservatives, pump manufacturers have developed different spray systems that avoid the need for preservatives. These systems use a collapsible bag, a movable piston, or a compressed gas to compensate for the emitted liquid volume. The solutions with a collapsible bag and a movable piston compensating for the emitted liquid volume offer the additional advantage that they can be emitted upside down, without the risk of sucking air into the dip tube and compromising the subsequent spray. This may be useful for some products where the patients are bedridden and where a head-down application is recommended. Another method used for avoiding preservatives is that the air that replaces the emitted liquid is filtered through an aseptic air filter. In addition, some systems have a ball valve at the tip to prevent contamination of the liquid inside the applicator tip. More recently, pumps have been designed with side-actuation. Pumps have been designed with a shorter tip to avoid contact with the sensitive mucosal surfaces. New designs to reduce the need for priming and re-priming, and pumps incorporating pressure point features to improve the dose reproducibility and dose counters and lock-out mechanisms for enhanced dose control and safety are available (see Aptar supply lists).

Traditional, simple single, bi-dose and multi-use metered-dose spray pumps require priming and some degree of overfill to maintain dose conformity for the labeled number of doses. They are well suited for drugs to be administered daily over a prolonged duration, but due to the priming procedure and limited control of dosing, unless a specialty device is selected, they are less suited for drugs with a narrow therapeutic window of time in which to use the device, particularly if they are not used often. For expensive drugs and drugs intended for single administration or sporadic use and where tight control of the dose and formulation is of importance, single-dose (UDS) or bi-dose spray (BDS) devices are preferred (on the World Wide Web at aptar.com). A simple variant of a single-dose spray device (MAD®) is offered by LMA (LMA, Salt Lake City, Utah, USA; on the World Wide Web at lmana.com). A nosepiece with a spray tip is fitted to a standard syringe. The liquid drug to be delivered is first drawn into the syringe and then the spray tip is fitted onto the syringe. This device has been used in academic studies to deliver, for example, a topical steroid in patients with chronic rhinosinusitis and in a vaccine study. A pre-filled device based on the same principle for one or two doses (Accuspray®, Becton Dickinson Technologies, Research Triangle Park, N.C., USA; on the World Wide Web at bdpharma.com) is used to deliver the influenza vaccine FluMist® (on the World Wide Web at flumist.com), approved for both adults and children in the US market. A similar device for two doses was marketed by a Swiss company for delivery of another influenza vaccine a decade ago.

Pre-primed single- and bi-dose devices are also available, and consist of a reservoir, a piston, and a swirl chamber (see, e.g., the UDS UnitDose® and BDS BiDose® devices from Aptar, formerly Pfeiffer). The spray is formed when the liquid is forced out through the swirl chamber. These devices are held between the second and the third fingers with the thumb on the actuator. A pressure point mechanism incorporated in some devices secures reproducibility of the actuation force and emitted plume characteristics. Currently, marketed nasal migraine drugs like Imitrex® (on the World Wide Web at gsk.com) and Zomig® (on the World Wide Web at az.com; Pfeiffer/Aptar single-dose device), the marketed influenza vaccine Flu-Mist (on the World Wide Web at flumist.com; Becton Dickinson single-dose spray device), and the intranasal formulation of naloxone for opioid overdose rescue, Narcan Nasal® (on the World Wide Web at narcan.com; Adapt Pharma) are delivered with this type of device.

In some embodiments, the 90% confidence interval for dose delivered per actuation is about 2%. In some embodiments, the 95% confidence interval for dose delivered per actuation is ±about 2.5%.

Historically, intranasal administration of drugs in large volume, such as from syringes adapted with mucosal atomizer devices (MADs), has encountered difficulty due to the tendency of some of the formulation to drip back out of the nostril or down the nasopharynx. Accordingly, in some embodiments, upon nasal delivery of said pharmaceutical formulation to said patient, less than about 20% of said pharmaceutical formulation leaves the nasal cavity via drainage into the nasopharynx or externally. In some embodiments, upon nasal delivery of said pharmaceutical formulation to said patient, less than about 10% of said pharmaceutical formulation leaves the nasal cavity via drainage into the nasopharynx or externally. In some embodiments, upon nasal delivery of said pharmaceutical formulation to said patient, less than about 5% of said pharmaceutical formulation leaves the nasal cavity via drainage into the nasopharynx or externally.

Current container closure system designs for inhalation spray drug products include both pre-metered and device-metered presentations using mechanical or power assistance and/or energy from patient inspiration for production of the spray plume. Pre-metered presentations contain previously measured doses or a dose fraction in some type of units (e.g., single or multiple blisters or other cavities) that are subsequently inserted into the device during manufacture or by the patient before use. Typical device-metered units have a reservoir containing formulation sufficient for multiple doses that are delivered as metered sprays by the device itself when activated by the patient.

With aseptic techniques, the use of preservatives may not be required in pre-primed devices, but overfill is required resulting in a waste fraction similar to the metered-dose, multi-dose sprays. To emit 100 μL, a volume of 125 μL is filled in the device (Pfeiffer/Aptar single-dose device) used for the intranasal migraine medications Imitrex® (sumatriptan) and Zomig® (zolmitriptan) and about half of that for a bi-dose design. Sterile drug products may be produced using aseptic processing or terminal sterilization. Terminal sterilization usually involves filling and sealing product containers under high-quality environmental conditions. Products are filled and sealed in this type of environment to minimize the microbial and particulate content of the in-process product and to help ensure that the subsequent sterilization process is successful. In most cases, the product, container, and closure have low bioburden, but they are not sterile. The product in its final container is then subjected to a sterilization process such as heat, irradiation, or chemical (gas). In an aseptic process, the drug product, container, and closure are first subjected to sterilization methods separately, as appropriate, and then brought together. Because there is no process to sterilize the product in its final container, it is critical that containers be filled and sealed in an efficient quality environment. Aseptic processing involves more variables than terminal sterilization. Before aseptic assembly into a final product, the individual parts of the final product generally can be subjected to various sterilization processes. For example, glass containers are subjected to dry heat; rubber closures are subjected to moist heat; and liquid dosage forms are subjected to filtration. Each of these manufacturing processes requires validation and control.

Devices recited herein may employ any of the pharmaceutical formulations, and are useful in the methods disclosed herein.

Accordingly, provided herein are devices adapted for nasal delivery of a pharmaceutical formulation to a patient, including a reservoir with a therapeutically effective amount of the compound of formula (I). In some embodiments, the compound of formula (I) is the only pharmaceutically active compound in the pharmaceutical formulation. In some embodiments, the volume of the pharmaceutical formulation in the reservoir is not more than about 140 μL.

In some embodiments, the volume of the pharmaceutical formulation in the reservoir is above about 125 μL and less than about 140 μL.

In some embodiments, about 100 μL of the pharmaceutical formulation in the reservoir is delivered to the patient in one actuation.

In some embodiments, about 100 μL of the pharmaceutical formulation in the reservoir is delivered to the patient in one actuation and includes less than about 2.5 mg of the compound of formula (I). In some embodiments, about 100 μL of the pharmaceutical formulation in the reservoir is delivered to the patient in one actuation and includes about 0.5 mg to about 2.5 mg of the compound of formula (I). In some embodiments, about 100 μL of the pharmaceutical formulation in the reservoir is delivered to the patient in one actuation and includes about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, or about 2.5 mg of the compound of formula (I).

In some embodiments, the nasal spray formulation further includes one or more excipients selected from water, EDTA, and sodium chloride. In some embodiments, the nasal spray formulation further includes benzalkonium chloride.

In some embodiments, about 100 μL of the liquid nasal spray formulation in the reservoir is delivered to the patient in one actuation and includes the compound of formula (I), dodecylmaltoside or benzalkonium chloride or a combination of dodecylmaltoside and benzalkonium chloride, EDTA, and NaCl.

In some embodiments, the nasal spray formulation is substantially free of antimicrobial preservatives.

In some embodiments, the nasal spray formulation further includes a compound which acts as a preservative, absorption enhancer and/or a cationic surfactant; an isotonicity agent; a stabilizing agent; and an amount of acid or base sufficient to achieve a pH of about 3.5 to about 6.0. The use of absorption enhancers, such as alkylsaccharides, cyclodextrins, and chitosans may increase the rate at which the compound of formula (I) is absorbed. In general, absorption enhancers provide improved pharmacokinetic outcomes such as increased Cₘₐₓ, reduced Tₘₐₓ, and dose proportionality compared to both intramuscular formulations and intranasal formulations that do not contain an absorption enhancer. Without being bound to any theory, such absorption enhancers typically operate by affecting two primary mechanisms for nasal absorption: paracellular transport via opening of tight junctions between cells, and transcellular transport or transcytosis through cells via vesicle carriers.

In some embodiments, the nasal spray formulation is any one of the liquid spray formulations as described herein.

Some absorption enhancing excipients can alter the paracellular and/or transcellular pathways, others can extend residence time in the nasal cavity or prevent metabolic changes. Without an absorption enhancer, the molecular-weight limit for nasal absorption is about 1 kDa, while administration of drugs in conjunction with absorption enhancers can enable the absorption of molecules from 1-30 kDa. Intranasal administration of most absorption enhancers, however, can cause nasal mucosa damage. Maggio, J Excipients and Food Chem. 5(2):100-12, 2014. Examples of absorption enhancers include aprotinin, benzalkonium chloride, benzyl alcohol, capric acid, ceramides, cetylpyridinium chloride, chitosan, cyclodextrins, deoxycholic acid, decanoyl carnitine, EDTA, glycocholic acid, glycodeoxycholic acid, glycofurol, glycosylated sphingosines, glycyrrhetinic acids, 2-hydroxypropyl-β-cyclodextrin, laureth-9, lauric acid, lauroyl carnitine, lauryl sulfate, lysophosphatidylcholine, menthol, poloxamer 407, poloxamer F68, poly-L-arginine, polyoxyethylene-9-lauryl ether, polysorbate 80, propylene glycol, quillaia saponin, salicylic acid, β-sitosterol-β-D-glucoside, sucrose cocoate, taurocholic acid, taurodeoxycholic acid, taurodihydrofusidic acid, and alkylsaccharides, such as dodecyl maltoside, tetradecyl maltoside and sucrose dodecanoate.

In some embodiments, the device is filled with the nasal spray formulation using sterile filling.

In some embodiments, the nasal spray formulation is chemically storage-stable for about twelve months at about 25° C. and about 60% relative humidity and about six months at about 40° C. and about 75% relative humidity.

In some embodiments, the compound of formula (I) is delivered as an aqueous solution, aqueous suspension, aqueous emulsion, non-aqueous solution, non-aqueous suspensions, non-aqueous emulsion, a solution with halogenated hydrocarbon propellant(s), or as a dry powder. In some embodiments, aqueous formulations are sprayed into the nostril. In some embodiments, aqueous formulations are aerosolized by liquid nebulizers employing either hydraulic or ultrasonic atomization. In some embodiments, non-aqueous formulations are sprayed into the nostril. In some embodiments, non-aqueous formulations are aerosolized by liquid nebulizers employing either hydraulic or ultrasonic atomization. Propellant-based systems may use suitable pressurized metered-dose inhalers (pMDIs). Dry powders may use dry powder inhaler devices (DPIs), which are capable of dispersing the drug substance effectively.

Propellants typically used include chlorofluorocarbons, hydrochlorofluorocarbons, hydrofluorocarbons, hydrocarbons, and compressed gases.

In some embodiments, the compound of formula (I) is delivered as a nasal aerosol produced by a nasal pressurized metered-dose inhalers (pMDIs). In some embodiments, the pMDI is a hydrofluroalkane (HFA)-based pMDI for nasal use. Like spray pumps, nasal pMDIs produce a localized deposition on the anterior non-ciliated epithelium of the nasal vestibule and in the anterior parts of the narrow nasal valve, but due to quick evaporation of the spray delivered with a pMDI, noticeable “drip-out” may be less of an issue.

In some embodiments, the compound of formula (I) is delivered with a nebulizer. Nebulizers use compressed gasses (air, oxygen, and nitrogen) or ultrasonic or mechanical power to break up medical solutions and suspensions into small aerosol droplets that can be directly inhaled into the nose. The smaller particles and slow speed of the nebulized aerosol increase penetration to the target sites in the middle and superior meatuses and the paranasal sinuses.

In some embodiments, the compound of formula (I) is delivered with a pulsating aerosol generated via a perforated vibrating membrane. In some embodiments, the pulsation membrane nebulizer is VibrENT (PARI Pharma GmbH). In some embodiments, the compound of formula (I) is delivered with a pulsating aerosol in combination with breathing techniques.

In some embodiments, the compound of formula (I) is delivered with Bi-Directional® delivery technology (e.g. Bi-Directional® Exhalation Delivery Systems (EDS); OptiNose).

In some embodiments, the compound of formula (I) is delivered with an atomizer. In some embodiments, the atomizer is a handheld battery-driven atomizer intended for nasal drug delivery. In some embodiments, the atomizer atomizes liquids by producing a vortical flow on the droplets as they exit the device. Such devices include the ViaNase® atomizer (by Kurve Technology Inc., Lynnwood, Wash., USA). In some embodiments, the atomizer is a nasal atomizer driven by highly pressurized nitrogen gas.

In some embodiments, the compound of formula (I) is delivered with a nasal powder device. In some embodiments, the nasal powder device is a nasal powder inhaler, nasal powder sprayer, or nasal powder insufflator. Powder sprayers typically have a compressible compartment to provide a pressure that when released creates a plume of powder particles fairly similar to that of a liquid spray. Breath-actuated inhalers require the user to use his or her own breath to inhale the powder into the nostril from a blister or capsule. Nasal insufflator devices consist of a mouthpiece and a nosepiece that are fluidly connected. Delivery occurs when the subject exhales into the mouthpiece to close the velum, and the airflow carries the powder particles into the nose through the device nosepiece.

In some embodiments, the nasal powder inhaler is a blister based powder inhaler. Typically, the blister is pierced before use and the device nosepiece placed into one nostril. The subject closes the other nostril with the finger and inhales the powder into the nose. Representative devises include BiDose®/Prohaler®, and Twin-lizer®.

Representative nasal powder sprayers include, but are not limited to, UnidoseDP®, Fit-lizer®, Monopowder®, SoluVent®)

In some embodiments, the nasal powder sprayer is a capsule-based, single-dose powder devices. In one such embodiment, the capsule-based, single-dose powder device consists of a chamber that cuts off the top and bottom of the capsule when inserted. A plastic chamber is compressed by hand, compressed air passes through a one-way valve and the capsule during actuation, and the powder is emitted.

In some embodiments, the nasal powder sprayer consists of an air-filled compartment that is compressed until a pin ruptures a membrane to release pressure that emits a plume of powder.

In some embodiments, the nasal powder sprayer consists of a plunger that when pressed creates a positive pressure that ruptures a membrane to expel the powder.

In some embodiments, the nasal powder insufflator requires the subject to blow into one end of the tube while the other end is inserted into the vestibule of the nostril.

In some embodiments, the compound of formula (I) is delivered with a breath-powered Bi-Directional® delivery device. A breathpowered Bi-Directional® nasal delivery device utilizes the exhaled breath to deliver the drug into the nose. Breath-powered Bi-Directional® devices consist of a mouthpiece and a sealing nosepiece with an optimized frusto-conical shape and comfortable surface that mechanically expands the first part of the nasal valve. The user slides a sealing nosepiece into one nostril until it forms a seal with the flexible soft tissue of the nostril opening, at which point, it mechanically expands the narrow slit-shaped part of the nasal triangular valve. The user then exhales through an attached mouthpiece. When exhaling into the mouthpiece against the resistance of the device, the soft palate (or velum) is automatically elevated by the positive oropharyngeal pressure, isolating the nasal cavity from the rest of the respiratory system. Owing to the sealing nosepiece, the dynamic pressure that is transferred from the mouth through the device to the nose further expands the slit-like nasal passages. This “breath-powered” mechanism enables release of liquid or powder particles into an air stream that enters one nostril, passes entirely around the nasal septum, and exits through the opposite nostril. Actuation of drug release in devices employing this approach use manual triggering or mechanisms automatically triggered by flow and/or pressure.

i. Single-Dose Devices

In some embodiments, the device is a single-dose device, wherein the nasal spray formulation is present in one reservoir, and wherein the therapeutically effective amount of the compound of formula (I) is delivered essentially by one actuation of the device.

Also provided herein is a single-use, pre-primed device adapted for nasal delivery of a pharmaceutical formulation to a patient by one actuation of the device into one nostril of the patient, having a single reservoir comprising about 100 μL of a liquid nasal spray formulation as disclosed herein.

In some embodiments, the device is actuatable with one hand.

In some embodiments, the delivery time is less than about 30 seconds. In some embodiments, the delivery time is less than about 25 seconds. In some embodiments, the delivery time is less than about 20 seconds. In some embodiments, the delivery time is less than about 15 seconds.

In some embodiments, the 90% confidence interval for dose delivered per actuation is ±about 2%. In some embodiments, the 95% confidence interval for dose delivered per actuation is ±about 2.5%.

In some embodiments, upon nasal delivery of the formulation to the patient, less than about 20%, less than about 15%, less than about 10%, or less than about 5%, of the formulation leaves the nasal cavity via drainage into the nasopharynx or externally, as provided above.

In some embodiments, the nasal spray formulation is chemically storage-stable for about twelve months at about 25° C. and about 60% relative humidity and/or about six months at about 40° C. and about 75% relative humidity.

ii. Bi-Dose Devices

In some embodiments, said device is a bi-dose device, wherein a first volume of said formulation is present in a first reservoir and a second volume of said formulation is present in a second reservoir, and wherein said therapeutically effective amount is delivered essentially by a first actuation of said device into a first nostril of said patient and a second actuation of said device into a second nostril of said patient.

In some embodiments, said first volume and said second volume combined is equal to not more than about 400 μL.

In some embodiments, about 100 μL of said first volume of said formulation is delivered by said first actuation.

In some embodiments, about 100 μL of said second volume of said formulation is delivered by said second actuation.

In some embodiments, said bi-dose device is actuatable with one hand.

In some embodiments, the delivery time is less than about 30 seconds. In some embodiments, the delivery time is less than about 25 seconds. In some embodiments, the delivery time is less than about 20 seconds. In some embodiments, the delivery time is less than about 15 seconds.

In some embodiments, the 90% confidence interval for dose delivered per actuation is about 2%. In some embodiments, the 95% confidence interval for dose delivered per actuation is ±about 2.5%.

In some embodiments, upon nasal delivery of the formulation to the patient, less than about 20%, less than about 15%, less than about 10%, or less than about 5%, of the formulation leaves the nasal cavity via drainage into the nasopharynx or externally.

D. Other Nasal Formulations

Nasal formulations, including an active agent, a compound of formula (I), can be in other forms, for example 1) Mucoadhesive drug delivery system (e.g., pectin, chitosan, or chitosan-poloxamer 188 as a mucoadhesive agent); 2) Nose-to-brain drug delivery by nanoparticles (e.g., chitosan and poly(lactic-co-glycolic acid) (PLGA) microspheres); and 3) Intranasal gels as an alternative to sprays.

Mucoadhesive drug delivery systems are delivery systems which utilize the property of bioadhesion of certain polymers (pectin, chitosan, or chitosan-poloxamer 188), which become adhesive on hydration and hence can be used for targeting a drug to a particular region of the body (e.g., nasal) for extended periods of time. Mucoadhesive drug delivery system (or formulation) includes a mucoadhesive agent (e.g., pectin, chitosan, or chitosan-poloxamer 188). On contact with the nasal mucosa, the formulation forms a gel and modulates the absorption of a drug (e.g., a compound of formula (I)) while limiting nasal drip or runoff. See References 11-12 under VII. REFERENCES.

The blood-brain barrier and the blood-cerebrospinal fluid barrier are major obstacles in central nervous system (CNS) drug delivery, since they block most molecules from entering the brain. Nose-to-brain delivery is a minimally invasive drug administration pathway, which bypasses the blood-brain barrier as the drug is directed from the nasal cavity to the brain. Intranasal drug delivery is very beneficial because it avoids first-pass metabolism and achieves a greater concentration of drugs in the central nervous system (CNS) at a low dose. The formulations suitable for the nose-to-brain delivery can include nanoparticles (NPs), microemulsions, in situ gel, etc. See References 13-15 under VII. REFERENCES.

Intranasal gels (e.g., in situ-based gels) can bypass the blood-brain barrier, deliver the therapeutics to the desired site, reduce peripheral toxicity and control drug release kinetics. See References 16-17 under VII. REFERENCES. Intranasal gels can be delivered by suitable nasal applicators, for example mono-dose device Lecticula from MetP Pharma AG. Examples of commercial products of nasal gels include Natesto, which is a no-drip, testosterone-containing gel.

### IV. Compounds

The present invention provides a compound for use in nasal formulations for the treatment of ADHD or a cognitive dysfunction disease or disorder. The compounds as defined and described herein, are represented by formula (I):

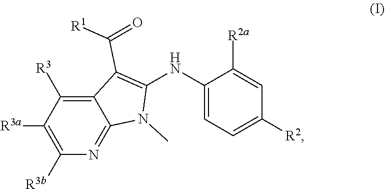

or stereoisomer, mixture of stereoisomers, and/or a pharmaceutically acceptable salt thereof, wherein:
R¹ is —OR⁴, —NR⁵R^{5a}, or —N(OR^{5b})R^{5a};
R² is halo, C₁-C₆ alkyl, —S—C₁-C₆ alkyl, C₃-C₈ cycloalkyl, C₂-C₆ alkenyl, or C₂-C₆ alkynyl;
R^{2a} is halo or C₁-C₆ alkyl;
R³, R^{3a}, and R^{3b} are independently hydrogen, halo, C₁-C₆ alkyl, or C₁-C₆ alkoxy;
R⁴ is C₁-C₆ alkyl, C₃-C₈ cycloalkyl, C₃-C₈ cycloalkyl-C₁-C₆ alkyl, C₁-C₆ hydroxyalkyl, or C₁-C₆ alkoxy-C₁-C₆ alkyl;
R⁵ is hydrogen, C₁-C₆ alkyl, C₃-C₈ cycloalkyl, C₃-C₈ cycloalkyl-C₁-C₆ alkyl, C₁-C₆ hydroxyalkyl, or C₁-C₆ alkoxy-C₁-C₆ alkyl;
R^{5a} is hydrogen or C₁-C₆ alkyl; and
R^{5b} is hydrogen, C₁-C₆ alkyl, C₃-C₈ cycloalkyl, C₃-C₈ cycloalkyl-C₁-C₆ alkyl, C₁-C₆ hydroxyalkyl, or C₁-C₆ alkoxy-C₁-C₆ alkyl.

In some embodiments, the cycloalkyl group provided in formula (I) is a saturated monocyclic C₃-C₈ cycloalkyl. In some embodiments, the C₃-C₈ cycloalkyl group, as alone or as part of C₃-C₈ cycloalkyl-C₁-C₆ alkyl is cyclopropyl or cyclobutyl. In some embodiments, the C₃-C₈ cycloalkyl group, as alone or as part of C₃-C₈ cycloalkyl-C₁-C₆ alkyl, is unsubstituted.

In some embodiments, R³, R^{3a}, and R^{3b} are each independently hydrogen, halo, or C₁-C₆ alkoxy. In some embodiments, R³, R^{3a}, and R^{3b} are each independently hydrogen or C₁-C₆ alkoxy. In some embodiments, R³, R^{3a}, and R^{3b} are each independently hydrogen, fluoro, or methoxy.

In some embodiments, R³ is hydrogen.

In some embodiments, R^{3a} is hydrogen, halo, or C₁-C₆ alkoxy. In some embodiments, R^{3a} is hydrogen. In some embodiments, R^{3a} is halo. In some embodiments, R^{3a} is fluoro, chloro, bromo, or iodo. In some embodiments, R^{3a} is fluoro. In some embodiments, R^{3a} is C₁-C₆ alkoxy. In some embodiments, R^{3a} is methoxy, ethoxy, propoxy, iso-propoxy, butoxy, 2-butoxy, iso-butoxy, sec-butoxy, tert-butoxy, pentoxy, or hexoxy. In some embodiments, R^{3a} is methoxy.

In some embodiments, R^{3b} is hydrogen.

In some embodiments, R³, R^{3a}, and R^{3b} are each hydrogen. In some embodiments, R³ and R^{3b} are each hydrogen and R^{3a} is halo or C₁-C₆ alkoxy. In some embodiments, R³ and R^{3b} are each hydrogen and R^{3a} is fluoro or methoxy. In some embodiments, R³ and R^{3b} are each hydrogen and R^{3a} is fluoro. In some embodiments, R³ and R^{3b} are each hydrogen and R^{3a} is methoxy.

In some embodiments, the compound is represented by formula (Ia):

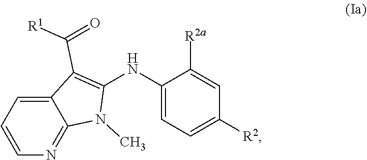

wherein R¹, R², and R^{2a} are as defined and described herein.

In some embodiments of formula (I) or (Ia), R¹ is —OR⁴. In some embodiments, R⁴ is C₁-C₆ alkyl. In some embodiments, R⁴ is C₁-C₃ alkyl. In some embodiments, R⁴ is C₃-C₈ cycloalkyl. In some embodiments, R⁴ is C₃-C₆ cycloalkyl. In some embodiments, R⁴ is C₃-C₈ cycloalkyl-C₁-C₆ alkyl. In some embodiments, R⁴ is C₃-C₆ cycloalkyl-C₁-C₆ alkyl. In some embodiments, R⁴ is cyclopropyl, cyclobutyl, cyclopropyl-C₁-C₃ alkyl, or cyclobutyl-C₁-C₃ alkyl. In some embodiments, R⁴ is cyclopropylmethyl. In some embodiments, R⁴ is C₁-C₆ hydroxyalkyl. In some embodiments, R⁴ is C₁-C₆ monohydroxyalkyl. In some embodiments, R⁴ is C₁-C₆ dihydroxyalkyl. In some embodiments, R⁴ is HOCH₂—C₁-C₅ alkyl. In some embodiments, R⁴ is C₁-C₃ hydroxyalkyl. In some embodiments, R⁴ is C₁-C₃ monohydroxyalkyl. In some embodiments, R⁴ is C₁-C₃ dihydroxyalkyl. In some embodiments, R⁴ is HOCH₂—C₁-C₂ alkyl. In some embodiments, R⁴ is CH₂CH₂OH. In some embodiments, R⁴ is CH₂CH(OH)CH₂OH.

In some embodiments of formula (I) or (Ia), R¹ is selected from the group consisting of:

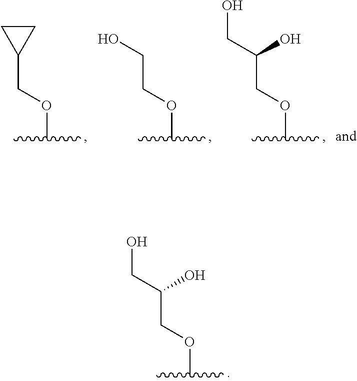 [SMILES: *OCC1CC1 ; *OCCO ; *OC[C@H](O)CO ; *OC[C@@H](O)CO]

In some embodiments of formula (I) or (Ia), R¹ is —NR⁵R^{5a}. In some embodiments, R⁵ is hydrogen. In some embodiments, R⁵ is C₁-C₆ alkyl. In some embodiments, R⁵ is C₁-C₃ alkyl. In some embodiments, R⁵ is C₃-C₈ cycloalkyl. In some embodiments, R⁵ is C₃-C₆ cycloalkyl. In some embodiments, R⁵ is C₃-C₈ cycloalkyl-C₁-C₆ alkyl. In some embodiments, R⁵ is C₃-C₆ cycloalkyl-C₁-C₆ alkyl. In some embodiments, R⁵ is cyclopropyl, cyclobutyl, cyclopropyl-C₁-C₃ alkyl, or cyclobutyl-C₁-C₃ alkyl. In some embodiments, R⁵ is cyclopropylmethyl. In some embodiments, R⁵ is C₁-C₆ hydroxyalkyl. In some embodiments, R⁵ is C₁-C₆ monohydroxyalkyl. In some embodiments, R⁵ is C₁-C₆ dihydroxyalkyl. In some embodiments, R⁵ is HOCH₂—C₁-C₅ alkyl. In some embodiments, R⁵ is C₁-C₃ hydroxyalkyl. In some embodiments, R⁵ is C₁-C₃ monohydroxyalkyl. In some embodiments, R⁵ is C₁-C₃ dihydroxyalkyl. In some embodiments, R⁵ is HOCH₂—C₁-C₂ alkyl. In some embodiments, R⁵ is CH₂CH₂OH. In some embodiments, R⁵ is CH₂CH(OH)CH₂OH.

In some embodiments of formula (I) or (Ia), R¹ is —NR⁵R^{5a}; R^{5a} is hydrogen; and R⁵ is as defined and described herein. In some embodiments, R¹ is —NR⁵R^{5a}; R^{5a} is C₁-C₆ alkyl; and R⁵ is as defined and described herein. In some embodiments, R¹ is —NR⁵R^{5a}; R^{5a} is C₁-C₃ alkyl; and R⁵ is as defined and described herein.

In some embodiments of formula (I) or (Ia), R1 is selected from the group consisting of:

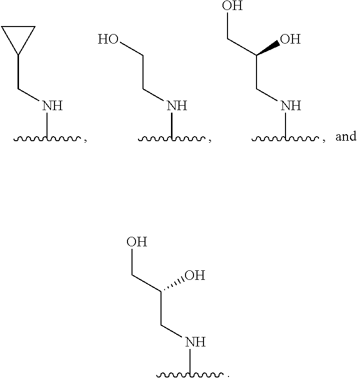 [SMILES: *NCC1CC1 ; *NCCO ; *NC[C@H](O)CO ; *NC[C@@H](O)CO]

In some embodiments of formula (I) or (Ia), R¹ is —N(OR^{5b})R^{5a}. In some embodiments, R^{5b} is hydrogen. In some embodiments, R^{5b} is C₁-C₆ alkyl. In some embodiments, R^{5b} is C₁-C₃ alkyl. In some embodiments, R^{5b} is C₃-C₈ cycloalkyl. In some embodiments, R^{5b} is C₃-C₆ cycloalkyl. In some embodiments, R^{5b} is C₃-C₈ cycloalkyl-C₁-C₆ alkyl. In some embodiments, R^{5b} is C₃-C₆ cycloalkyl-C₁-C₆ alkyl. In some embodiments, R^{5b} is cyclopropyl, cyclobutyl, cyclopropyl-C₁-C₃ alkyl, or cyclobutyl-C₁-C₃ alkyl. In some embodiments, R^{5b} is cyclopropylmethyl. In some embodiments, R^{5b} is C₁-C₆ hydroxyalkyl. In some embodiments, R^{5b} is C₁-C₆ monohydroxyalkyl. In some embodiments, R^{5b} is C₁-C₆ dihydroxyalkyl. In some embodiments, R^{5b} is HOCH₂—C₁-C₅ alkyl. In some embodiments, R^{5b} is C₁-C₃ hydroxyalkyl. In some embodiments, R^{5b} is C₁-C₃ monohydroxyalkyl. In some embodiments, R^{5b} is C₁-C₃ dihydroxyalkyl. In some embodiments, R^{5b} is HOCH₂—C₁-C₂ alkyl. In some embodiments, R^{5b} is CH₂CH₂OH. In some embodiments, R^{5b} is CH₂CH(OH)CH₂OH.

In some embodiments of formula (I) or (Ia), R¹ is —N(OR^{5b})R^{5a}; R^{5a} is hydrogen; and R^{5b} is as defined and described herein. In some embodiments, R¹ is —N(OR^{5b})R^{5a}; R^{5a} is C₁-C₆ alkyl; and R^{5b} is as defined and described herein. In some embodiments, R¹ is —N(OR^{5b})R^{5a}; R^{5a} is C₁-C₃ alkyl; and R^{5b} is as defined and described herein.

In some embodiments of formula (I) or (Ia), R¹ is selected from the group consisting of:

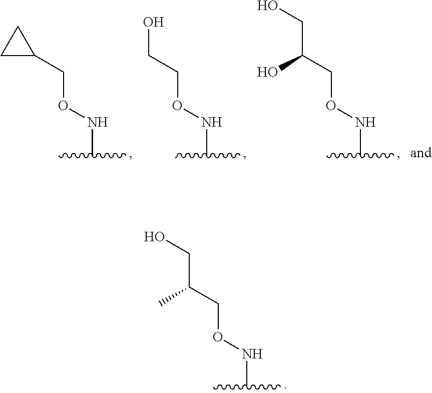

In some embodiments of formula (I) or (Ia), R² is halo, C₁-C₆ alkyl, —S—C₁-C₆ alkyl, C₃-C₈ cycloalkyl, C₂-C₆ alkenyl, or C₂-C₆ alkynyl. In some embodiments, R² is halo or C₁-C₆ alkyl. In some embodiments, R² is halo, —CH₃, —SCH₃, C₂-C₃ alkenyl, or C₂-C₃ alkynyl.

In some embodiments of formula (I) or (Ia), R² is halo. In some embodiments, R² is fluoro. In some embodiments, R² is iodo. In some embodiments, R² is chloro. In some embodiments, R² is bromo.

In some embodiments of formula (I) or (Ia), R² is C₁-C₆ alkyl. In some embodiments, R² is C₁-C₃ alkyl. In some embodiments, R² is methyl.

In some embodiments of formula (I) or (Ia), R² is —S—C₁-C₆ alkyl. In some embodiments, R² is —S—C₁-C₃ alkyl. In some embodiments, R² is —SCH₃.

In some embodiments of formula (I) or (Ia), R² is C₃-C₈ cycloalkyl. In some embodiments, R² is cyclopropyl.

In some embodiments of formula (I) or (Ia), R² is C₂-C₆ alkenyl. In some embodiments, R² is C₂-C₄ alkenyl. In some embodiments, R² is vinyl (ethenyl), propenyl, isopropenyl, 1-butenyl, 2-butenyl, isobutenyl, or butadienyl. In some embodiments, R² is vinyl.

In some embodiments of formula (I) or (Ia), R² is C₂-C₆ alkynyl. In some embodiments, R² is C₂-C₃ alkynyl. In some embodiments, R² is acetylenyl or propynyl. In some embodiments, R² is acetylenyl.

In some embodiments of formula (I) or (Ia), R^{2a} is halo or C₁-C₃ alkyl. In some embodiments, R^{2a} is halo or CH₃. In some embodiments, R^{2a} is fluoro or CH₃. In some embodiments, R^{2a} is iodo or CH₃. In some embodiments, R^{2a} is chloro or CH₃. In some embodiments, R^{2a} is bromo or CH₃.

In some embodiments of formula (I) or (Ia), R^{2a} is halo. In some embodiments, R^{2a} is fluoro. In some embodiments, R^{2a} is iodo. In some embodiments, R^{2a} is chloro. In some embodiments, R^{2a} is bromo.

In some embodiments of formula (I) or (Ia), R^{2a} is C₁-C₆ alkyl. In some embodiments, R^{2a} is C₁-C₃ alkyl. In some embodiments, R^{2a} is CH₃.

In some embodiments of formula (I) or (Ia), R² and R^{2a} are each halo. In some embodiments, R² is halo and R^{2a} is C₁-C₆ alkyl. In some embodiments, R² is C₁-C₆ alkyl and R^{2a} is halo. In some embodiments, R² is —S—C₁-C₆ alkyl and R^{2a} is halo. In some embodiments, R² is —SCH₃ and R^{2a} is halo. In some embodiments, R² is C₃-C₈ cycloalkyl and R^{2a} is halo. In some embodiments, R² is cyclopropyl and R^{2a} is halo. In some embodiments, R² is C₂-C₆ alkenyl and R^{2a} is halo. In some embodiments, R² is C₂-C₆ alkynyl and R^{2a} is halo. In some embodiments, R² is acetylenyl and R^{2a} is halo. In some embodiments, R² and R^{2a} are each independently fluoro, chloro, bromo, or iodo. In some embodiments, R² is iodo and R^{2a} is fluoro. In some embodiments, R² is halo and R^{2a} is —CH₃. In some embodiments, R² is bromo and R^{2a} is —CH₃. In some embodiments, R² is iodo and R^{2a} is —CH₃. In some embodiments, R² is —SCH₃ and R^{2a} is fluoro. In some embodiments, R² is acetylenyl and R^{2a} is fluoro.

In some embodiments of formula (I) or (Ia), the compound is represented by formula (Ib):

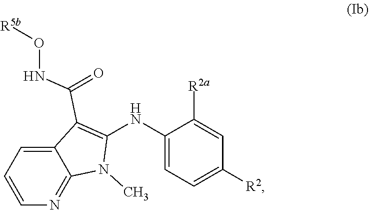

wherein R², R^{2a}, and R^{5b} are defined and described herein.

In some embodiments of formula (Ib), R² is iodo and R^{2a} is fluoro. In some embodiments, R² is iodo and R^{2a} is methyl. In some embodiments, R² is acetylenyl and R^{2a} is fluoro. In some embodiments, R² is —SCH₃ and R^{2a} is fluoro. In some embodiments of the above structures, R² is —SCH₃ and R^{2a} is methyl.

In some embodiments, the compound is represented by formula (Ib-1):

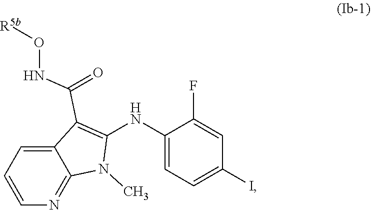

wherein R^{5b} is defined and described herein.

In some embodiments of formula (Ib) or (Ib-1), R^{5b} is cyclopropylmethyl. In some embodiments, R^{5b} is C₁-C₃ monohydroxyalkyl. In some embodiments, R^{5b} is C₁-C₃ dihydroxyalkyl. In some embodiments, R^{5b} is HOCH₂—C₁-C₂ alkyl. In some embodiments, R^{5b} is CH₂CH₂OH. In some embodiments, R^{5b} is CH₂CH(OH)CH₂OH.

In some embodiments of formula (Ib) or (Ib-1), R^{5b} is selected from the group consisting of:

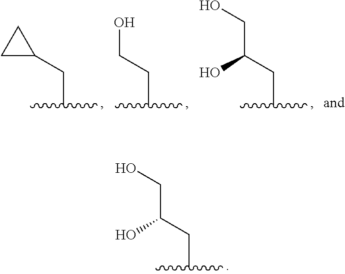

In some embodiments, the compound is represented by the formula:

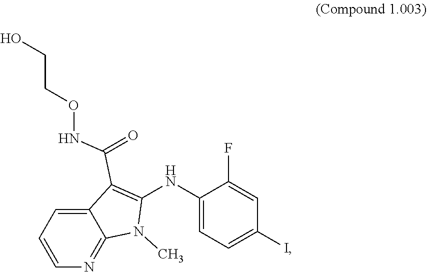 [SMILES: Cn1c(Nc2ccc(I)cc2F)c(C(=O)NOCCO)c2cccnc21]

having the name of 2-((2-fluoro-4-iodophenyl)amino)-N-(2-hydroxyethoxy)-1-methyl-1H-pyrrolo[2,3-b]pyridine-3-carboxamide.

Exemplified compounds of formula (I) are listed in Table 1.

TABLE 1
Compounds of formula (I)

| No. | Structure |
|---|---|
| 1.001 | 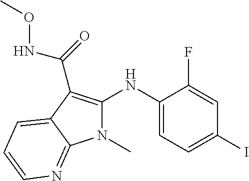 [SMILES: CONC(=O)c1c(Nc2ccc(I)cc2F)n(C)c2ncccc12] |
| 1.002 | 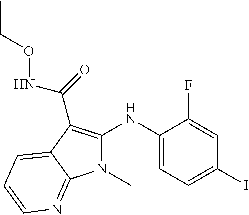 [SMILES: CCONC(=O)c1c(Nc2ccc(I)cc2F)n(C)c2ncccc12] |
| 1.003 | 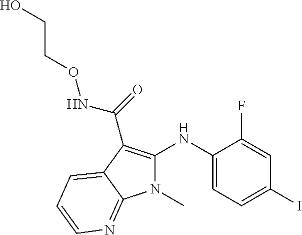 [SMILES: Cn1c(Nc2ccc(I)cc2F)c(C(=O)NOCCO)c2cccnc21] |
| | 2-((2-fluoro-4- |
| | iodophenyl)amino)-N-(2- |
| | hydroxyethoxy)-1-methyl-1H- |
| | pyrrolo[2,3-b]pyridine-3- |
| | carboxamide |
| 1.004 | 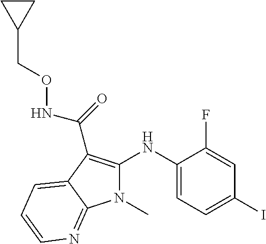 [SMILES: Cn1c(Nc2ccc(I)cc2F)c(C(=O)NOCC2CC2)c2cccnc21] |
| 1.005 | 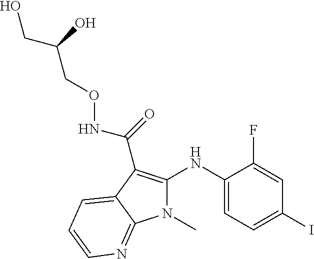 [SMILES: Cn1c(Nc2ccc(I)cc2F)c(C(=O)NOC[C@H](O)CO)c2cccnc21] |
| 1.006 | 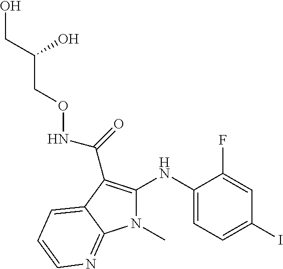 [SMILES: Cn1c(Nc2ccc(I)cc2F)c(C(=O)NOC[C@@H](O)CO)c2cccnc21] |
| 1.007 | 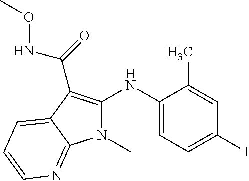 [SMILES: CONC(=O)c1c(Nc2ccc(I)cc2C)n(C)c2ncccc12] |
| 1.008 | 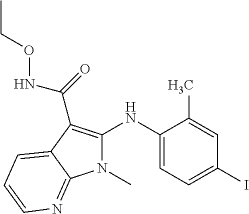 [SMILES: CCONC(=O)c1c(Nc2ccc(I)cc2C)n(C)c2ncccc12] |
| 1.009 | 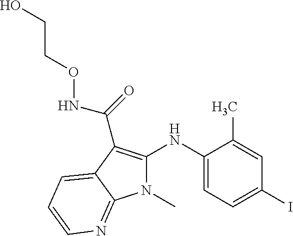 [SMILES: Cc1cc(I)ccc1Nc1c(C(=O)NOCCO)c2cccnc2n1C] |
| 1.010 | 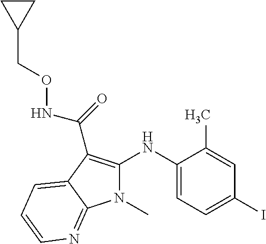 [SMILES: Cc1cc(I)ccc1Nc1c(C(=O)NOCC2CC2)c2cccnc2n1C] |
| 1.011 | 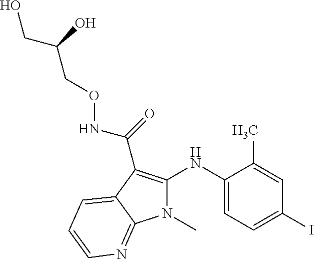 [SMILES: Cc1cc(I)ccc1Nc1c(C(=O)NOC[C@H](O)CO)c2cccnc2n1C] |
| 1.012 | 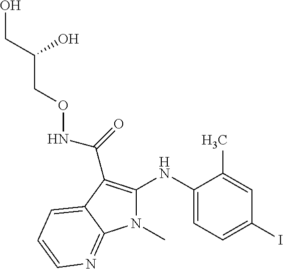 [SMILES: Cc1cc(I)ccc1Nc1c(C(=O)NOC[C@@H](O)CO)c2cccnc2n1C] |
| 1.013 | 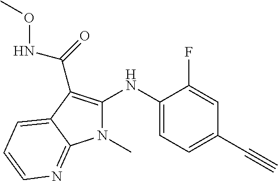 [SMILES: C#Cc1ccc(Nc2c(C(=O)NOC)c3cccnc3n2C)c(F)c1] |
| 1.014 | 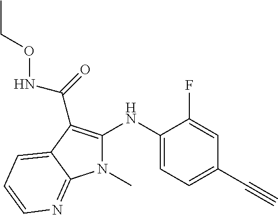 [SMILES: C#Cc1ccc(Nc2c(C(=O)NOCC)c3cccnc3n2C)c(F)c1] |
| 1.015 | 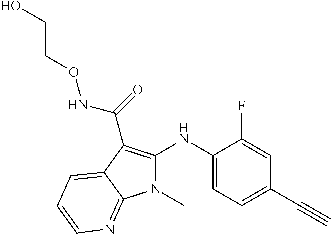 [SMILES: C#Cc1ccc(Nc2c(C(=O)NOCCO)c3cccnc3n2C)c(F)c1] |
| 1.016 | 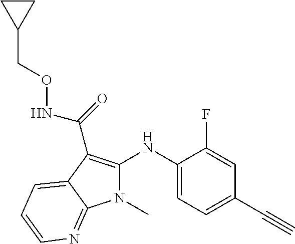 [SMILES: C#Cc1ccc(Nc2c(C(=O)NOCC3CC3)c3cccnc3n2C)c(F)c1] |
| 1.017 | 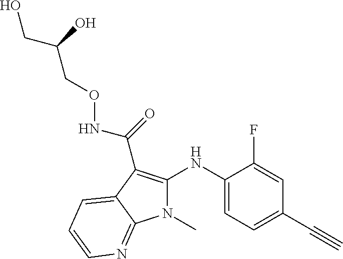 [SMILES: C#Cc1ccc(Nc2c(C(=O)NOC[C@H](O)CO)c3cccnc3n2C)c(F)c1] |
| 1.018 | 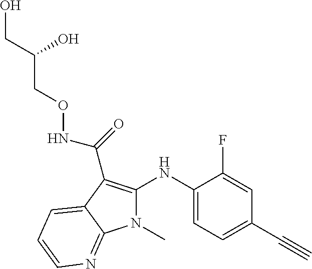 [SMILES: C#Cc1ccc(Nc2c(C(=O)NOC[C@@H](O)CO)c3cccnc3n2C)c(F)c1] |
| 1.019 | 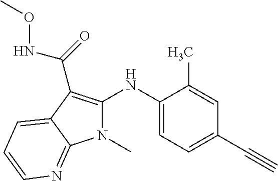 [SMILES: C#Cc1ccc(Nc2c(C(=O)NOC)c3cccnc3n2C)c(C)c1] |
| 1.020 | 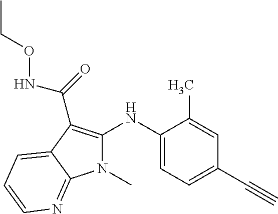 [SMILES: C#Cc1ccc(Nc2c(C(=O)NOCC)c3cccnc3n2C)c(C)c1] |
| 1.021 | 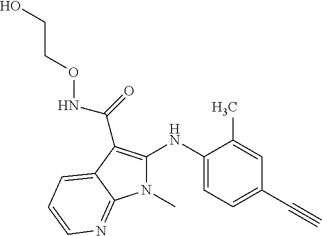 [SMILES: C#Cc1ccc(Nc2c(C(=O)NOCCO)c3cccnc3n2C)c(C)c1] |
| 1.022 | 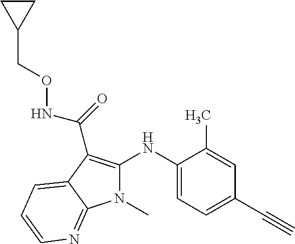 [SMILES: C#Cc1ccc(Nc2c(C(=O)NOCC3CC3)c3cccnc3n2C)c(C)c1] |
| 1.023 | 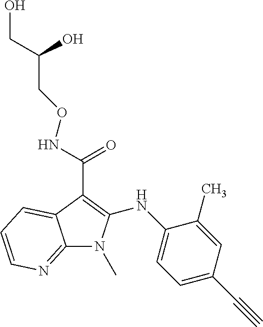 [SMILES: C#Cc1ccc(Nc2c(C(=O)NOC[C@H](O)CO)c3cccnc3n2C)c(C)c1] |
| 1.024 | 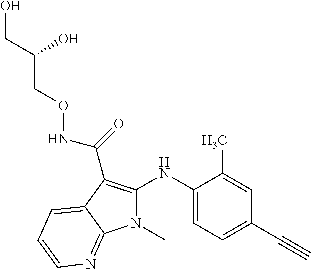 [SMILES: C#Cc1ccc(Nc2c(C(=O)NOC[C@@H](O)CO)c3cccnc3n2C)c(C)c1] |
| 1.025 | 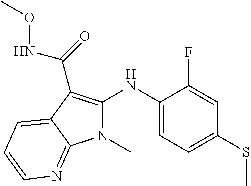 |
| 1.026 | 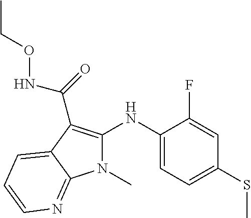 [SMILES: CCONC(=O)c1c(Nc2ccc(SC)cc2F)n(C)c2ncccc12] |
| 1.027 | 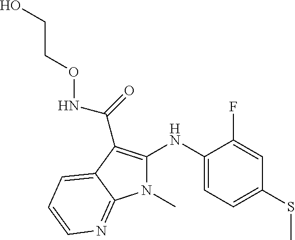 [SMILES: CSc1ccc(Nc2c(C(=O)NOCCO)c3cccnc3n2C)c(F)c1] |
| 1.028 | 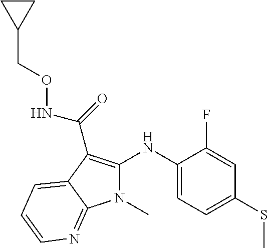 [SMILES: CSc1ccc(Nc2c(C(=O)NOCC3CC3)c3cccnc3n2C)c(F)c1] |
| 1.029 | 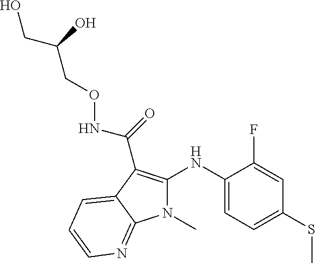 [SMILES: CSc1ccc(Nc2c(C(=O)NOC[C@H](O)CO)c3cccnc3n2C)c(F)c1] |
| 1.030 | 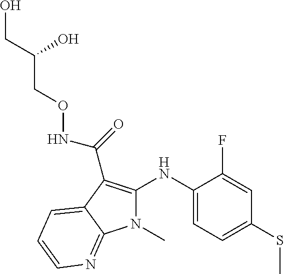 [SMILES: CSc1ccc(Nc2c(C(=O)NOC[C@@H](O)CO)c3cccnc3n2C)c(F)c1] |
| 1.031 | 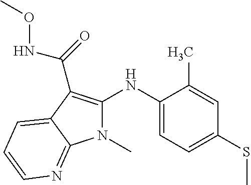 |
| 1.032 | 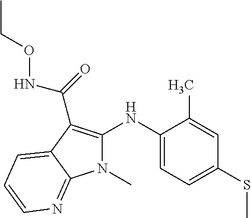 [SMILES: CCONC(=O)c1c(Nc2ccc(SC)cc2C)n(C)c2ncccc12] |
| 1.033 | 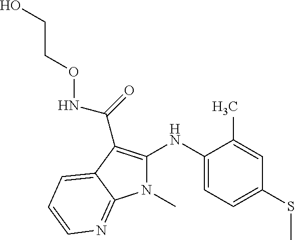 [SMILES: CSc1ccc(Nc2c(C(=O)NOCCO)c3cccnc3n2C)c(C)c1] |
| 1.034 | 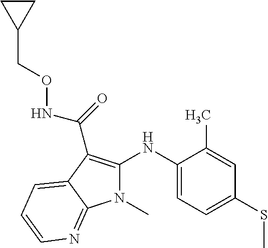 [SMILES: CSc1ccc(Nc2c(C(=O)NOCC3CC3)c3cccnc3n2C)c(C)c1] |
| 1.035 | 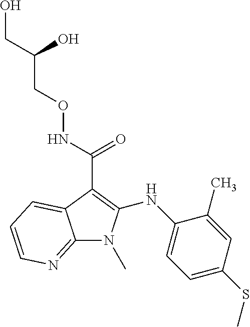 [SMILES: CSc1ccc(Nc2c(C(=O)NOC[C@H](O)CO)c3cccnc3n2C)c(C)c1] |
| 1.036 | 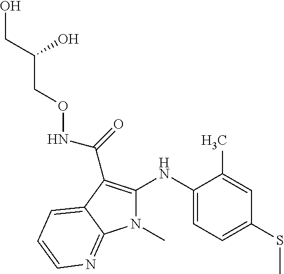 [SMILES: CSc1ccc(Nc2c(C(=O)NOC[C@@H](O)CO)c3cccnc3n2C)c(C)c1] |
| 1.037 | 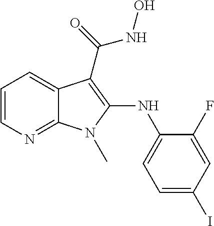 [SMILES: Cn1c(Nc2ccc(I)cc2F)c(C(=O)NO)c2cccnc21] |
| 1.038 | 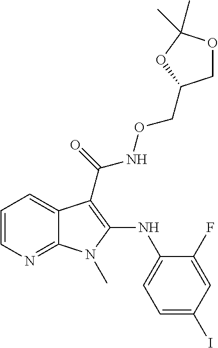 [SMILES: Cn1c(Nc2ccc(I)cc2F)c(C(=O)NOC[C@H]2COC(C)(C)O2)c2cccnc21] |
| 1.039 | 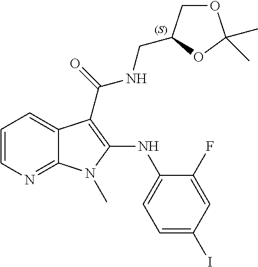 |
| 1.040 | 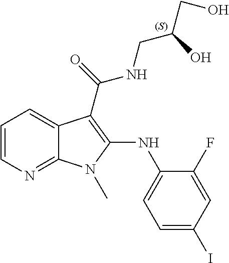 |
| 1.041 | 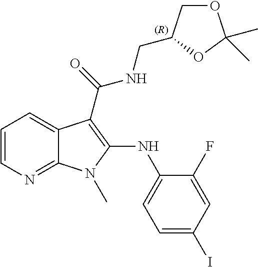 |
| 1.042 | 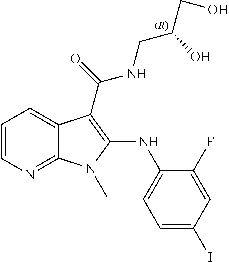 |
| 1.043 | 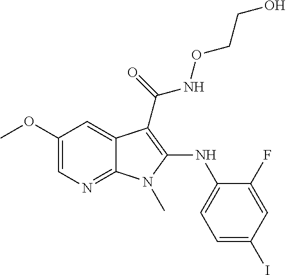 [SMILES: COc1cnc2c(c1)c(C(=O)NOCCO)c(Nc1ccc(I)cc1F)n2C] |
| 1.044 | 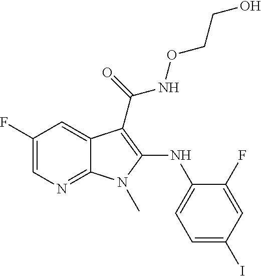 [SMILES: Cn1c(Nc2ccc(I)cc2F)c(C(=O)NOCCO)c2cc(F)cnc21] |
| 1.045 | 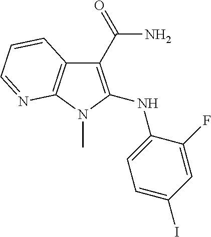 [SMILES: Cn1c(Nc2ccc(I)cc2F)c(C(N)=O)c2cccnc21] |

The compounds of formula (I) can be prepared according to PCT/US2018/033547, the entirety of which is incorporated herein by reference for all purposes.

The compounds of the present invention may exist as salts. The present invention includes such salts. Examples of applicable salt forms include hydrochlorides, hydrobromides, sulfates, methanesulfonates, nitrates, maleates, acetates, citrates, fumarates, tartrates (eg (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present invention contain both basic and acidic functionalities that allow the compounds to be converted into either base or acid addition salts.

Other salts include acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, which is incorporated herein by reference.

Pharmaceutically acceptable salts includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the enantiomers, racemates, diastereomers, tautomers, geometric isomers, stereoisometric forms that may be defined, in terms of absolute stereochemistry, as I- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active I- and (S)-, or (D)- and (L)-isomers may be prepared using chiral synthons or chiral reagents, or resolved using conventional techniques.

Isomers include compounds having the same number and kind of atoms, and hence the same molecular weight, but differing in respect to the structural arrangement or configuration of the atoms.

It will be apparent to one skilled in the art that certain compounds of this invention may exist in tautomeric forms, all such tautomeric forms of the compounds being within the scope of the invention. Tautomer refers to one of two or more structural isomers which exist in equilibrium and which are readily converted from one isomeric form to another.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, the compounds of the present invention may also contain unnatural proportions of atomic isotopes at one or more of the atoms that constitute such compounds. For example, the compounds of the present invention may be labeled with radioactive or stable isotopes, such as for example deuterium (²H), tritium (³H), iodine-125 (¹²⁵I), fluorine-18 (¹⁸F), nitrogen-15 (¹⁵N), oxygen-17 (¹⁷O), oxygen-18 (¹⁸O), carbon-13 (¹³C), or carbon-14 (¹⁴C). All isotopic variations of the compounds of the present invention, whether radioactive or not, are encompassed within the scope of the present invention.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

### V. Methods—Indications

Provided herein are methods of treating ADHD or a cognitive dysfunction disease or disorder, in a subject having a neurofibromatosis and in need of treatment, the method including administering intranasally to said subject a nasal spray formulation comprising a compound represented by formula (I) (see above). In some embodiments, the neurofibromatosis is selected from the group consisting of neurofibromatosis type-1, neurofibromatosis type-2, or schwannomatosis.

As described herein, by administering compounds of formula (I) nasally, the present inventors have discovered that these compounds can be useful in the treatment of ADHD, or a cognitive dysfunction disease or disorder in a subject having a neurofibromatosis. The neurofibromatosis can be neurofibromatosis type-1, neurofibromatosis type-2, or schwannomatosis.

Cognitive dysfunction disease or disorders include conditions that impair a subject ability to perform normal high-level brain functions. These can include impairment in the ability to learn and remember information, organize, organize, plan, problem-solve, focus, maintain and shift attention.

In some embodiments, the cognitive dysfunction is a neurodegenerative disease or disorder. Neurodegenerative disease are characterized by degenerative changes in neuronal cells that cause nervous system loss in function. In some embodiments neurodegenerative diseases are those causing problems with movement or sensation. In some embodiments, neurodegenerative diseases are those affecting memory or related to dementia

In some embodiments, the cognitive dysfunction is a neurodevelopmental disorder. Neurodevelopmental disorders include conditions involving abnormal neurodevelopment such as attentional and perceptual processing, executive function, inhibitory control. In some embodiments, the neurodevelopmental disorder is a learning disability. Learning disabilities include, but are not limited to, difficulty with reading, writing, math and memory. In some embodiments, the neurodevelopmental disorder is an attention deficit disorder. Attention deficit disorder include ADD, ADHD and related clinical diagnoses. In some embodiments, the neurodevelopmental disorder is a seizure disorder. In some embodiments, the seizure disorder is epilepsy.

Exemplary conditions, conditions treatable in accordance with the formulations and methods provided herein include, but are not necessarily limited to ADHD, learning disorders, attention deficit disorder (ADD), Alper's disease, obsessive-compulsive disorder (OCD), Alzheimer's disease, dementia with Lewy bodies, Parkinson's disease, or Huntington's disease.

### VI. Kits

Also provided are kits for use in methods of treatment of ADHD or a cognitive dysfunction disease or disorder, in a subject in need thereof having a neurofibromatosis. In some embodiments, the neurofibromatosis is selected from the group consisting of neurofibromatosis type-1, neurofibromatosis type-2, or schwannomatosis. The kits can include a nasal spray formulation including a compound of formula (I) provided herein, optionally a second agent or composition, and instructions providing information to a health care provider regarding usage for treating a responsive disorder or disease. Instructions may be provided in printed form or in the form of an electronic medium such as a floppy disc, CD, or DVD, or in the form of a website address where such instructions may be obtained. A unit dose of a compound or a nasal spray formulation provided herein, or an optional second agent or composition, can include a dosage such that when administered to a subject, a therapeutically or prophylactically effective plasma level of the compound can be maintained in the subject for at least 1 day.

In some embodiments, suitable packaging is provided. As used herein, “packaging” includes a solid matrix or material customarily used in a system and capable of holding within fixed limits a compound provided herein and/or an optional second agent suitable for administration to a subject. Such materials include glass and plastic (e.g., polyethylene, polypropylene, and polycarbonate) bottles, vials, paper, plastic, and plastic-foil laminated envelopes and the like. If e-beam sterilization techniques are employed, the packaging should have sufficiently low density to permit sterilization of the contents.

### VI. References

1. Blüthgen, N., van Bentum, M., Merz, B. et al. Profiling the MAPK/ERK dependent and independent activity regulated transcriptional programs in the murine hippocampus in vivo. Sci Rep 7, 45101 (2017).
2. Provenzano G, Pangrazzi L, Poli A, Pernigo M, Sgadò P, Genovesi S, Zunino G, Berardi N, Casarosa S, Bozzi Y. Hippocampal dysregulation of neurofibromin-dependent pathways is associated with impaired spatial learning in engrailed 2 knock-out mice. J Neurosci. 2014 Oct. 1; 34(40):13281-8.
3. Provenzano G, Pangrazzi L, Poli A, Pernigo M, Sgadò P, Genovesi S, Zunino G, Berardi N, Casarosa S, Bozzi Y. Hippocampal dysregulation of neurofibromin-dependent pathways is associated with impaired spatial learning in engrailed 2 knock-out mice. J Neurosci. 2014 Oct. 1; 34(40):13281-8. DOI: 10.1523/JNEUROSCI.2894-13.2014.
4. Provenzano G, Pangrazzi L, Poli A, Pernigo M, Sgadò P, Genovesi S, Zunino G, Berardi N, Casarosa S, Bozzi Y. Hippocampal dysregulation of neurofibromin-dependent pathways is associated with impaired spatial learning in engrailed 2 knock-out mice. J Neurosci. 2014 Oct. 1; 34(40):13281-8. DOI: 10.1523/JNEUROSCI.2894-13.2014.
5. Blüthgen N, van Bentum M, Merz B. et al. Profiling the MAPK/ERK dependent and independent activity regulated transcriptional programs in the murine hippocampus in vivo. Sci Rep 7, 45101 (2017).
6. Blüthgen N, van Bentum M, Merz B et al. Profiling the MAPK/ERK dependent and independent activity regulated transcriptional programs in the murine hippocampus in vivo. Sci Rep 7, 45101 (2017).
7. Blüthgen N, van Bentum M, Merz B et al. Profiling the MAPK/ERK dependent and independent activity regulated transcriptional programs in the murine hippocampus in vivo. Sci Rep 7, 45101 (2017).
8. Kang M, Lee Y. The impact of RASopathy-associated mutations on CNS development in mice and humans. Molecular Brain (2019) 12:96. DOI:10.1186/s13041-019-0517-5.
9. Ryu H, Kang M, Park J, Park S, Lee Y. Enriched expression of NF1 in inhibitory neurons in both mouse and human brain. Molecular Brain (2019) 12:60. DOI:10.1186/s13041-019-0481-0.
10. Cui Y, Costa R, Murphy G, Elgersma Y, Zhu Y, Gutmann D, Parada L, Mody I, Silva A. Neurofibromin regulation of ERK signaling modulates GABA release and learning. Cell. 2008 Oct. 31; 135(3): 549-560. DOI: 10.1016/j.cell.2008.09.060.
11. Fisher A, Watling M, Smith A, Knight A. Pharmacokinetic comparisons of three nasal fentanyl formulations; pectin, chitosan and chitosan-poloxamer 188. International Journal of Clinical Pharmacology and Therapeutics, 1 Feb. 2010, 48(2):138-145. DOI: 10.5414/cpp48138. PMID: 20137766.
12. Fisher A, Watling M, Smith A, Knight A. Pharmacokinetics and relative bioavailability of fentanyl pectin nasal spray 100-800 μg in healthy volunteers. International Journal of Clinical Pharmacology and Therapeutics, 1 Dec. 2010, 48(12):860-867. DOI: 10.5414/cpp48860. PMID: 21084042.
13. Wang et al. Nose-to-Brain Delivery. J Pharmacol Exp Ther 370:593-601, September 2019. DOI: 10.1124/jpet.119.258152.
14. Ong W Y, Shalini S M, Costantino L. Nose-to-brain drug delivery by nanoparticles in the treatment of neurological disorders. Curr Med Chem. 2014; 21(37):4247-56. DOI: 10.2174/0929867321666140716103130. PMID: 25039773.
15. Ansari M A, Chung I M, Rajakumar G, Alzohairy M A, Alomary M N, Thiruvengadam M, Pottoo F H, Ahmad N. Current Nanoparticle Approaches in Nose to Brain Drug Delivery and Anticancer Therapy—A Review. Curr Pharm Des. 2020; 26(11):1128-1137. DOI: 10.2174/1381612826666200116153912. PMID: 31951165.
16. Banks W A et al. Delivery of testosterone to the brain by intranasal administration: comparison to intravenous testosterone. J Drug Target. 2009 February; 17(2):91-7. DOI: 10.1080/10611860802382777. PMID: 19089688.
17. van Wingen G A et al. Testosterone increases amygdala reactivity in middle-aged women to a young adulthood level. Neuropsychopharmacology. 2009 February; 34(3):539-47.

### VIII. Examples

### Example 1: Preparation of a Nasal Formulation

The following Example describes the preparation of exemplary liquid nasal spray formulations of the present disclosure.

The liquid nasal spray formulations of the present invention can be prepared according to the procedure provided below. Reaction conditions, steps and reactants not provided in the procedure below would be apparent to, and known by, those skilled in the art.

Excipients (i.e., absorption enhancers, antioxidants, and/or the preservative) were aliquoted or weighted into individual vials to form a mixture. The compound of formula (I) (e.g., Compound 1.003) was added to the mixture to achieve a desired concentration or saturation. Then the viscosity regulating agent (e.g., HPC) were added accordingly. The pH was adjusted with 0.1 M citric acid in PEG-400 or sodium phosphate monobasic/sodium phosphate dibasic to about 6-7. Finally, a second addition of PEG-400 (or water) was used to titrate the formulation to 100% by weight. The vials were vortexed to mix and spin overnight. Afterwards, a viscosity and a visual inspection were immediately recorded, then stored at ambient conditions for 7 days.

Table 2 summarizes the preparation components and relative amounts used (wt/wt %) for three separate formulations.

TABLE 2
Exemplary Nasal Formulations

| | | Composition (wt/wt %) | | |
|---|---|---|---|---|
| Function | Component | Ex. A | Ex. B | Ex. C |
| API | Compound 1.003* | 2.34 | 2.34 | 2.34 |
| Stabilizing | Disodium EDTA | — | — | 0.20 |
| Agent | | | | |
| | Water | — | — | 30.00 |
| Absorption | S. R. PEG-400** | 47.11 | 66.84 | 20.00 |
| Enhancers | PEG-1450 | — | — | 5.00 |
| | Propylene glycol | — | — | 12.00 |
| | Transcutol HP | 45.00 | 25.00 | 25.00 |
| Antioxidant | Ascorbyl Palmitate | 0.05 | 0.05 | — |
| | Alpha tocopherol | 0.002 | 0.02 | — |
| | Butylated | — | — | 0.05 |
| | hydroxytoluene | | | |
| | (BHT) | | | |
| Viscosity | Hydroxypropyl- | 0.5 | 0.25 | — |
| Regulating | cellulose | | | |
| Agent | (HPC) (Klucel HF) | | | |
| Preservative | Phenoxyethanol | — | — | 1.00 |
| pH | Sodium phosphate | — | — | 0.55 |
| Adjustment | monobasic | | | |
| Agent | Sodium phosphate | — | — | Q.S. to |
| | dibasic | | | pH 6-7 |
| | 0.1M Citric Acid | Q.S. to | Q.S. to | — |
| | in SR PEG-400 | pH 6-7 | pH 6-7 | |
| Absorption | 2^{nd} addition of S.R. | Q.S. to | Q.S. to | — |
| Enhancers | PEG-400 | 100 | 100 | |
| | 2^{nd} addition of | — | — | Q.S. to |
| | water | | | 100 |

* The amount of Compound 1.003 added may be adjusted based on API purity/potency;
** Part of PEG-400 was adjusted to compensate the addition of the pH adjusting solution and final Q.S. 100; and
Abbreviations: S. R. - super refined; HP - high purity; and Q.S. - quantum satis

### Example 2: Penetration of Compound 1.003 into Brain Via Intranasal Administration

This study was conducted to investigate the potential for Compound 1.003 to penetrate into the brain following a single intranasal administration of a nasal formulation including the compound. Compound 1.003 was formulated at a dose strength of 2.3% according to Ex. A of Example 1.

### Objectives

A single group of female athymic mice each received a single intranasal administration of Compound 1.003. At intervals post dose the animals were sacrificed and samples of blood and brain were collected. The concentration of Compound 1.003 was determined in plasma and brain; and samples of brain were used for determination of the expression of phosphor-ERK (p-ERK).

### Test Animals

The study was conducted using the following animals:
Species: Mouse;
Strain: NCr mu/mu athymic nude mice from Charles River;
Sex: Female;
Age: 7 weeks old at the time of dosing;
Body weight: 20.1 to 27.0 g; and
Number used: 12

Animals were maintained at the experimental site according to local procedures. Pretreatment and during the study, animals received food and water ad libitum.

### Study Design

Overview: A single group of 12 athymic female mice each received a single intranasal administration of Compound 1.003 (Ex. A nasal formulation of Example 1). Three animals were sacrificed at each a 4 times post dose and sample of plasma and brain collected at necropsy. The concentration of Compound 1.003 was determined in plasma and brain by liquid chromatography with tandem mass spectrometry (LC-MS/MS) and the expression of p-ERK was determined in brain samples.

Dosing: Each animal received a single administration (50 μL) of the nasal formulation Ex. A including Compound 1.003. The dose was administration intranasally to each animal.

Sampling: Following administration of the nasal formulation Ex. A including Compound 1.003 to 12 female mice, 3 were sacrificed at each of 0.25, 0.5, 1 and 4 h post dose with the following samples collected:
Blood Collection: Collect full volume blood by terminal cardiac puncture under isoflurane anesthesia.
Process blood for Plasma: anti-coagulant—K₂EDTA, preservation—Frozen at −80° C., shipping condition −80° C. (dry ice). The samples were send out for the LC-MS/MS analysis of plasma concentration of Compound 1.003.
Brain Collection: Brain (divide into 2 parts at the mid sagittal plane); Part 1: preservation—snap frozen, ship at −80° C. (dry ice) for the LC-MS/MS analysis of Compound 1.003; and Part 2: preservation—fixed in 10% neutral buffered formalin for at least 24 h. Samples were then transferred after 24 h into 1.5 mL Eppendorf tubes containing 70% EtOH and stored ambient until shipment for the expression of p-ERK.

### Experimental Procedures

Bioanalytical Methods: Bioanalysis of rat plasma and brain samples for Compound 1.003 was performed using the LC-MS/MS analysis. For this study, the analysis was conducted using existing fit for purpose bioanalytical methods. Plasma samples were analyzed undiluted against a plasma standard curve (10 standards between 0.5 to 5000 ng/mL). Prior to analysis for concentration of compound 1.003, the brain samples were weighed, mixed with 5 volumes of water and then homogenised. Homogenised samples were then diluted 2× into plasma and then analysed against the plasma standard curve (final dilution 10×). All results were then corrected for the dilution factor.

Immunohistochemistry for p-ERK: Immunohistochemistry staining of murine brain sections for p-ERK was performed by HistoWiz Inc. (Brooklyn, N.Y.) using standard operating procedures and fully automated workflow. Samples were processed, embedded in paraffin, and sectioned at 4 m. Immunohistochemistry was performed on a Bond Rx autostainer (Leica Biosystems) with enzyme treatment (1:1000) using standard protocols. Antibodies used were rabbit p-ERK (Cell Signaling, 4307S, 1:100). Bond Polymer Refine anti-rabbit HRP Detection (Leica Biosystems) was used according to manufacturer's protocol. Sections were then counterstained with hematoxylin, dehydrated and film coverslipped using a TissueTek-Prisma and Coverslipper (Sakura). Whole slide scanning (40×) was performed on an Aperio AT2 (Leica Biosystems). The images were quantified using Halo image analysis software (Indica Labs) using CytoNuclear module.

### Results

Plasma: The concentration of Compound 1.003 in plasma following single intranasal administration of the nasal formulation Ex. A is shown in Table 3.

TABLE 3
Plasma Concentrations of Compound 1.003

| Time | Animal replicates | | | | | % |
|---|---|---|---|---|---|---|
| (h) | 1 | 2 | 3 | Mean | SD | CV |
| 0.25 | 1190 | 1230 | 568 | 996 | 371 | 37.3% |
| 0.5 | 757 | 1000 | 823 | 860 | 126 | 14.6% |
| 1.0 | 130 | 82.4 | 125 | 112 | 26.2 | 23.3% |
| 4.0 | 45.5 | 52.4 | 82.4 | 60.1 | 19.6 | 32.6% |

Following intranasal administration, there was a relatively rapid absorption of Compound 1.003 with the maximum plasma concentration (Cₘₐₓ) of 996 ng/mL measured at 15 min, the first time point. After the Cₘₐₓ, the systemic concentration dropped rapidly to 1 hour and then dropped slowly over the following 4 hours.

Brain: The concentration of Compound 1.003 in brain following single intranasal administration of the nasal formulation Ex. A is shown in Table 4.

TABLE 4
Brain Concentrations of Compound 1.003

| Time | Animal replicates | | | | | % |
|---|---|---|---|---|---|---|
| (h) | 1 | 2 | 3 | Mean | SD | CV |
| 0.250 | 405 | 375 | 280 | 353 | 65.3 | 18.5% |
| 0.500 | 297 | 513 | 383 | 398 | 109 | 27.3% |
| 1.00 | 188 | 205 | 144 | 179 | 31.5 | 17.6% |
| 4.00 | 166 | 46.5 | 51.9 | 88.1 | 67.5 | 76.6% |

The concentration of Compound 1.003 in the brain followed a similar profile to that in the plasma. The Cₘₐₓ in the brain was at 0.5 hour post dose after which the concentration declined at a slower rate than in the plasma but followed the same trend.

The brain:plasma ratio of Compound 1.003 is shown in Table 5. As the clearance from plasma was faster than from the brain, the ratio tended to increase over time, from 0.38 at 15 min up to 1.7 at 1 and 4 hour(s) post dose.

TABLE 5
Brain:plasma Concentration ratio of Compound 1.003

| Time | Animal number | | | | | | | | | | | | | | % |
|---|---|---|---|---|---|---|---|---|---|---|---|---|---|---|---|
| (h) | 1 | 2 | 3 | 4 | 5 | 6 | 7 | 8 | 9 | 10 | 11 | 12 | Mean | SD | CV |
| 0.250 | 0.34 | 0.30 | 0.49 | | | | | | | | | | 0.38 | 0.100 | 26.3% |
| 0.500 | | | | 0.39 | 0.51 | 0.47 | | | | | | | 0.46 | 0.061 | 13.3% |
| 1.00 | | | | | | | 1.45 | 2.49 | 1.15 | | | | 1.70 | 0.702 | 41.4% |
| 4.00 | | | | | | | | | | 3.65 | 0.89 | 0.63 | 1.72 | 1.673 | 97.2% |

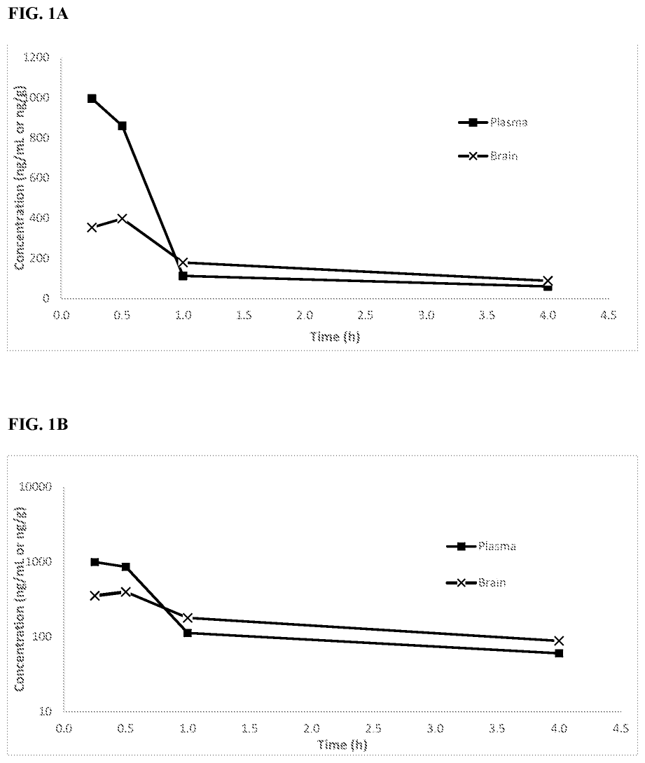

FIG. 1A and FIG. 1B show plasma and brain concentrations of Compound 1.003 in female mice following single intranasal administration of 50 μL of the nasal formulation Ex. A including 2.3% compound 1.003.

Immunohistochemical staining of p-ERK in mouse specimens 1, 2, 3 (15 min), and 10, 11, 12 (4 hours) detected robust nuclear p-ERK staining in the hippocampus consistent with known expression of p-ERK in rat hippocampus.

Summary: Following single intranasal administration, Compound 1.003 was detected in both brain and the systemic circulation with similar concentration in brain and plasma. A brain to plasma ratio of Compound 1.003 was from 0.38 at 15 min up to 1.7 at 1 and 4 hour(s) post dose. Immunohistochemistry detected p-ERK in sagittal brain sections of mouse hippocampus.

### CONCLUSION

Following intranasal administration, the nasal formulation Ex. A including 2.3% compound 1.003 is able to deliver drug into mouse brain tissue. Bioanalytical methods were developed to measure levels of Compound 1.003 in the brain. Immunohistochemistry assays for p-ERK can be employed for pharmacodynamic analysis of Compound 1.003 in mouse brain.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of treating ADHD or a cognitive dysfunction disease or disorder, in a subject having neurofibromatosis type-1, neurofibromatosis type-2, or schwannomatosis and in need of treatment, said method comprising administering intranasally to said subject a nasal spray formulation comprising a compound represented by formula (I): 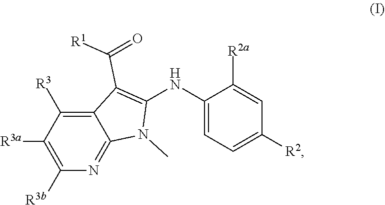
or stereoisomer, mixture of stereoisomers, and/or a pharmaceutically acceptable salt thereof,
wherein
R¹ is —OR⁴, —NR₅R^{5a}, or —N(OR^{5b})R^{5a};
R² is halo, C₁-C₆ alkyl, —S—C₁-C₆ alkyl, C₃-C₈ cycloalkyl, C₂-C₆ alkenyl, or C₂-C₆ alkynyl;
R^{2a} is halo or C₁-C₆ alkyl;
R³, R^{3a}, and R^{3b} are independently hydrogen, halo, C₁-C₆ alkyl, or C₁-C₆ alkoxy;
R⁴ is C₁-C₆ alkyl, C₃-C₈ cycloalkyl, C₃-C₈ cycloalkyl-C₁-C₆ alkyl, C₁-C₆ hydroxyalkyl, or C₁-C₆ alkoxy-C₁-C₆ alkyl;
R⁵ is hydrogen, C₁-C₆ alkyl, C₃-C₈ cycloalkyl, C₃-C₈ cycloalkyl-C₁-C₆ alkyl, C₁-C₆ hydroxyalkyl, or C₁-C₆ alkoxy-C₁-C₆ alkyl;
R^{5a} is hydrogen or C₁-C₆ alkyl; and
R^{5b} is hydrogen, C₁-C₆ alkyl, C₃-C₈ cycloalkyl, C₃-C₈ cycloalkyl-C₁-C₆ alkyl, C₁-C₆ hydroxyalkyl, or C₁-C₆ alkoxy-C₁-C₆ alkyl.

2. The method of claim 1, wherein the compound is represented by formula (Ib) or (Ib-1):

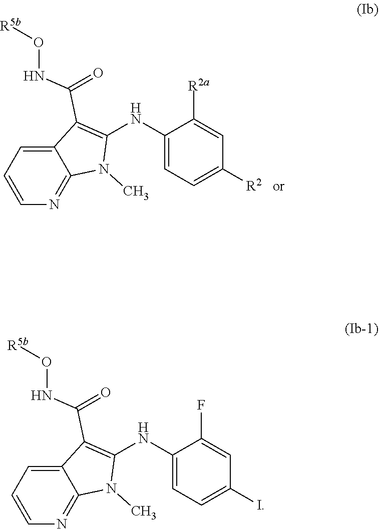

3. The method of claim 1, wherein the compound is represented by the formula:

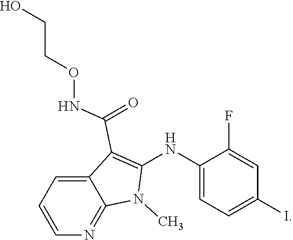 [SMILES: Cn1c(Nc2ccc(I)cc2F)c(C(=O)NOCCO)c2cccnc21]

4. The method of claim 1, wherein the compound is administered in a metered dose.

5. The method of claim 1, wherein the subject has neurofibromatosis type-1.

6. The method of claim 1, wherein the subject has neurofibromatosis type-2.

7. The method of claim 1, wherein the subject has schwannomatosis.

8. The method of claim 1, wherein the subject has been diagnosed with ADHD.

9. The method of claim 1, wherein the subject has been diagnosed with a cognitive dysfunction disease or disorder.

10. The method of claim 9, wherein the cognitive dysfunction disease or disorder is a neurodegenerative disease or disorder.

11. The method of claim 9, wherein the cognitive dysfunction disease or disorder is a neurodevelopmental disorder.

12. The method of claim 9, wherein the cognitive dysfunction disease or disorder is a learning disorder.

13. The method of claim 9, wherein the cognitive dysfunction disease or disorder is an attention deficit disorder.

14. The method of claim 9, wherein the cognitive dysfunction disease or disorder is epilepsy.

15. The method of claim 1, wherein the nasal spray formulation is delivered to the subject in one actuation of a nasal spray device.

16. The method of claim 15, wherein the nasal spray formulation is a liquid nasal spray formulation or a powdered nasal spray formulation.

17. The method of claim 15, wherein the nasal spray formulation is a liquid nasal spray formulation.

18. The method of claim 15, wherein the nasal spray formulation is a powdered nasal spray formulation.

19-27. (canceled)

28. A powdered nasal spray formulation, comprising a compound of formula (I) and carrier particles.

29. The powdered nasal spray formulation of claim 28, wherein the compound of formula (I) is present in an amount of from about 0.01% to about 5% or from about 0.01% to about 3% by weight.

30. A method of treating ADHD or a cognitive dysfunction disease or disorder, in a subject having neurofibromatosis type-1, neurofibromatosis type-2, or schwannomatosis and in need of treatment, said method comprising administering intranasally to the subject a nasal spray formulation of claim 28.

31. The method of claim 30, wherein the cognitive dysfunction disease or disorder is a learning disorder, an attention deficit disorder, or epilepsy.
